(19) United States
(12) Patent Application Publication
(10) Pub. No.: US 2004/0132164 A1
(43) Pub. Date: Jul. 8, 2004

(54) Methods and compositions for inhibiting adhesion by microorganisms

(76) Inventors: Ron J. Doyle, Louisville, KY (US); M. M. Cowan, Cincinnati, OH (US)

Correspondence Address: Genencor International, Inc., 925 Page Mill Road, Palo Alto, CA, 94034-1013, US

(21) Appl. No.: 10/741,740

(22) Filed: Dec. 18, 2003

Related U.S. Application Data
(63) Continuation of application No. 09/750,857, filed on Dec. 29, 2000, now abandoned.
(60) Provisional application No. 60/173,821, filed on Dec. 30, 1999.

(51) Int. Cl. C12N 1/20

(52) U.S. Cl. 435/252.3

(57) ABSTRACT

The present invention is directed generally to compositions and methods for enzymatic reduction of adhesion by a microorganism to cells, tissues, extracellular matrix, teeth, and/or dental prostheses. The compositions of the invention include pharmaceutical compositions and oral care compositions containing an enzyme that can reduce binding of a microbe to a cell, a tissue, or a surface. Suitable enzymes include a polyphenol oxidase and an asparaginase.

### FIELD OF THE INVENTION

The present invention is directed generally to compositions and methods for enzymatic reduction of adhesion by a microorganism to surfaces, such as cells, tissues, extracellular matrix, teeth, prostheses, and medical devices. The compositions of the invention include pharmaceutical compositions, oral care compositions, and cleaning compositions containing one or more enzymes that can reduce binding of a microbe to a cell, a tissue, or a surface. Suitable enzymes include polyphenol oxidase and asparaginase.

### BACKGROUND OF THE INVENTION

The emergence of drug-resistance among some pathogenic microorganisms necessitates a search for alternative methods to battle infections. Investigators have discovered and studied agents toxic to infecting microorganisms. Some investigators seek to fight disease by interrupting earlier stages of infection. These earlier stages include adhesion by a microorganism to a host tissue, which can be a prerequisite to establishing a harmfil infection. Reducing adhesion by a microorganism may prevent infection and, therefore, disease. Progress has been made in understanding adhesion by a microorganism. (Ofek, I. and Doyle R. J., Bacterial Adhesion to Cells and Tissues. Chapman and Hall, New York (1994)). Known strategies for reducing adhesion by a microorganism include using carbohydrate analogs to inhibit interactions between an adhesin molecule on a microorganism and a sugar on a host cell or tissue. (Zopf, D. et al., Adv. Exp. Med. Biol. 408:35-8 (1996)). These analogs can be part of a carbohydrate cocktail, which includes carbohydrates having various structures corresponding to one or more lectins of a microorganism. (Beuth, J. et al., Adv. Exp. Med. Biol. 408:51-56 (1996)). Manipulating gene regulation to prevent phenotype switching by the microorganism to an adhesion-plus variant provides yet another strategy for reducing adhesion by a microorganism. (Kahane, I. et al., Adv. Exp. Med. Biol. 408:107-111 (1996)).

Investigators have reported that, in the oral cavity, Streptococcus mutans attaches to glucans deposited on the tooth surface. (Koga, T. et al., J. Gen. Microbiol. 132:2873-2883 (1986)). Such attachment is believed to enhance the ability of S. mutans to metabolize dietary sucrose to acid, which then can destroy tooth enamel and eventually result in a carious lesion. S. mutans and other oral streptococci use a surface protein called glucan-binding lectin (GBL) to attach to surface-bound glucan. (Gibbons, R. J. et al., J. Bacteriol. 98:341-346 (1969)). Drake et al. developed an in vitro model system using soluble high-molecular weight dextrans and whole cell suspensions of S. cricetus to examine GBL binding. (Drake, D. et al., Infect. Immun. 56:1864-1872 (1988); Drake, D. et al., Infect. Immun. 56:2205-2207 (1988)). The glucan binding results in aggregation that is quantifiable with spectrophotometry.

Analysis of the active sites of several specific binding lectins from bacteria and viruses has demonstrated that tyrosine and/or asparagine residues are present at the active sites. However, to date, enzymatic modification of tyrosine or asparagine residues on an adhesin molecule of a microorganism has not been exploited to reduce adhesion by a microorganism. Given the prevalence of harm caused by infection with microorganisms, and the medical effort and cost devoted to fighting these infections, there is a need for additional and improved compositions and methods for fighting infection by microorganisms. There is also a need for additional and improved compositions and methods for reducing adhesion by microorganisms to and in animal tissues, and for reducing adhesion by microorganisms to dental prostheses.

### SUMMARY OF THE INVENTION

The present invention is directed to compositions and methods for enzymatic reduction of adhesion by one or more microorganisms to cells, tissues, extracellular matrix, teeth, prostheses, medical devices, and/or other surfaces. Preferred enzymes for use in the invention include polyphenol oxidase and asparaginase.

In one embodiment, the invention includes a method of reducing binding of a microorganism to a surface, including enzymatically modifying an adhesin, such as a carbohydrate binding site, on the microorganism. Preferred enzymatic modifications employ polyphenol oxidase and/or asparaginase. In another embodiment, the invention provides a method of reducing adhesion by a microorganism to mammalian tissues or cells. The method can include administering to the animal an effective amount of an enzyme, such as polyphenol oxidase and/or asparaginase, for inhibiting or abolishing such adhesion by a microorganism or microorganisms. Administration of the enzyme to the am mal can be accomplished by any method suitable for delivering an enzyme to the site of a microorganism at or in an animal tissue or cell. For example, administration of the enzyme to the animal can be oral or topical. Administration can optionally be targeted to mammalian tissues infected by tissue destroying pathogens, or to the nose, ear, vagina, skin, lungs or digestive tract of the mammal.

In another embodiment, the invention provides an oral care composition including an effective amount of an enzyme, such as polyphenol oxidase and/or asparaginase, for reducing adhesion by a microorganism to oral tissues or cells or to a dental prosthesis, and a, carrier suitable for an oral care composition. Oral care compositions of the invention include but are not limited to a mouthwash, a toothpaste, an implant, or a combination thereof, and may optionally be in the form of a solid, a semi-solid, a liquid, or an aerosol.

In yet another embodiment, the invention provides a method for reducing adhesion by a microorganism to mammalian oral tissues or cells or to a dental prosthesis. The method may include administering to a mammal's oral cavity an oral care composition including an effective amount of an enzyme, such as polyphenol oxidase and/or asparaginase, to reduce adhesion by a microorganism. Advantages of this method can include reducing adhesion by one or more microorganisms to teeth; reducing dental caries, plaque, or calculus; reducing co-aggregation of microorganisms; reducing pellicle formation, inhibiting glucosyltransferase, or a combination thereof.

Administration of the oral care composition to the mammal's oral cavity can be accomplished by any method suitable for delivering a composition to the oral cavity. For example, administration of the oral care composition can include rinsing with a liquid, applying a semisolid with a toothbrush, swab, or syringe, implanting a solid, or a combination thereof. Optionally, the oral care composition can be used to treat a dental prosthesis, either in the oral cavity or outside the oral cavity. Such a treatment can include applying to a dental prosthesis removed from a mammal's oral cavity an oral care composition including an effective amount of polyphenol oxidase, asparaginase and/or other enzyme(s) to reduce adhesion by a microorganism.

### BRIEF DESCRIPTION OF THE DRAWINGS

FIG. 1 illustrates the change in absorbance as a function of time for glucan aggregation of S. sobrinus, and prevention of this aggregation by polyphenol oxidase.

FIG. 2 illustrates polyphenol oxidase induced reversal of the glucan aggregation of S. sobrinus. The plots show the change in absorbance caused by the aggregation as a function of time. Arrows indicate the times at which polyphenol oxidase was added.

FIG. 3 illustrates an electrophoresis activity gel showing the effect of polyphenol oxidase on activity of glucosyltransferase-I and glucosyltransferase-S. Lanes 1 and 3 each show an untreated glucosyltransferase preparation. Lanes 2 and 4 each show a polyphenol oxidase treated glucosyltransferase preparation. 2.1 μg protein samples were loaded onto lanes 1 and 2; 0.2 μg were loaded onto lanes 3 and 4.

FIG. 4 illustrates inhibition by asparaginase of binding by E. coli to urinary epithelial cells (UECs).

FIG. 5 illustrates inhibition by polyphenol oxidase treatment of adhesion by type I fimbriated E. coli. Bacteria were treated with increasing concentrations of polyphenol oxidase (71, 141, or 282 u/ml) then incubated with UECs to allow for adhesion. Degree of adhesion is represented as a percentage based on the adhesion of untreated bacteria to UECs, which was set at 100%.

FIG. 6 illustrates inhibition by asparaginase treatment of adhesion by type I fimbriated E. coli. Bacteria were treated with increasing concentrations of asparaginase (1.25, 2.5, 5, or 10 u/ml) then incubated with UECs to allow for adhesion. Degree of adhesion is represented as a percentage based on the adhesion of untreated bacteria to UECs, which was set at 100%.

FIG. 7 illustrates inhibition by sequential treatments with polyphenol oxidase and asparaginase on the adhesion of type I fimbriated E. coli to UECs. Bacteria were treated with polyphenol oxidase (141 u/ml) followed by treatment with asparaginase (10 u/ml) or vice versa then incubated with UECs. Degree of adhesion is represented as a percentage based on the adhesion of untreated bacteria to UECs, which was set at 100%.

FIG. 8 illustrates the protective effects of mannose against action of polyphenol oxidase and asparaginase on the Fim H binding site, which was competitively blocked with mannose. Mannose (50 mM) was used to completely block the binding site. Degree of adhesion is represented as a percentage based on the adhesion of untreated bacteria to UECs, which was set at 100%.

FIG. 9 illustrates inhibition by polyphenol oxidase treatment of adhesion by P fimbriated E. coli. Bacteria were treated with increasing concentrations of polyphenol oxidase (71, 141, or 282 u/ml) then incubated with UECs to allow for adhesion. Degree of adhesion is represented as a percentage based on the adhesion of untreated bacteria to UECs, which was set at 100%.

FIG. 10 illustrates inhibition by asparaginase treatment of adhesion by P fimbriated E. coli. Bacteria were treated with increasing concentrations of asparaginase (2.5, 5, or 25 u/ml) then incubated with UECs to allow for adhesion. Degree of adhesion is represented as a percentage based on the adhesion of untreated bacteria to UECS, which was set at 100%.

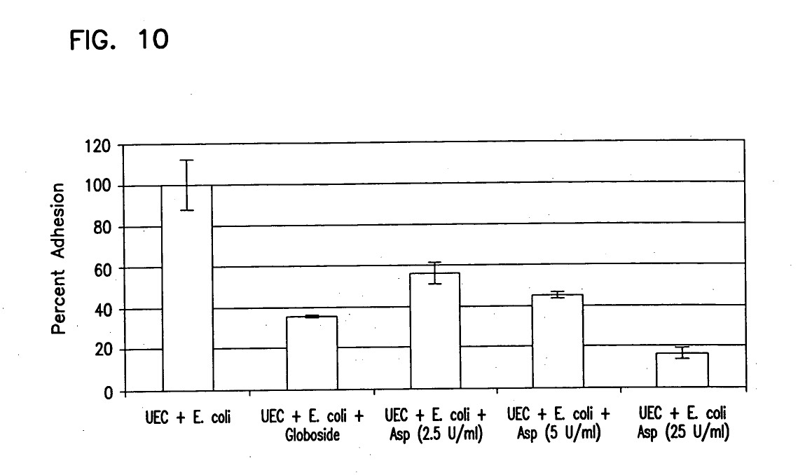

FIG. 11 illustrates inhibition by sequential enzymatic treatments on adhesion by P fimbriated E. coli to UECs. Bacteria were treated with polyphenol oxidase (141 u/ml) followed by treatment with asparaginase (10 u/ml) or vice versa then incubated with UECs. Degree of adhesion is represented as a percentage based on the adhesion of untreated bacteria to UECs, which was set at 100%.

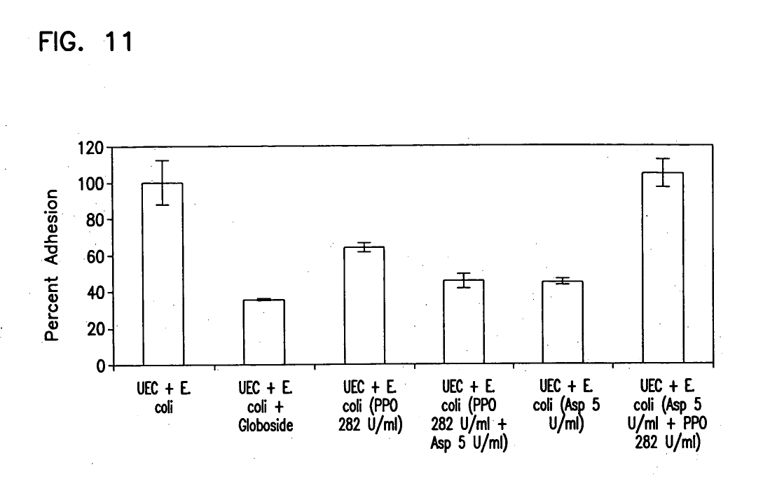

FIG. 12 illustrates the protective effects of globoside against action of polyphenol oxidase and asparaginase on the Pap G binding site, which was competitively blocked with globoside. Globoside was used to completely block the binding site. Degree of adhesion is represented as a percentage based on the adhesion of untreated bacteria to UECs, which was set at 100%.

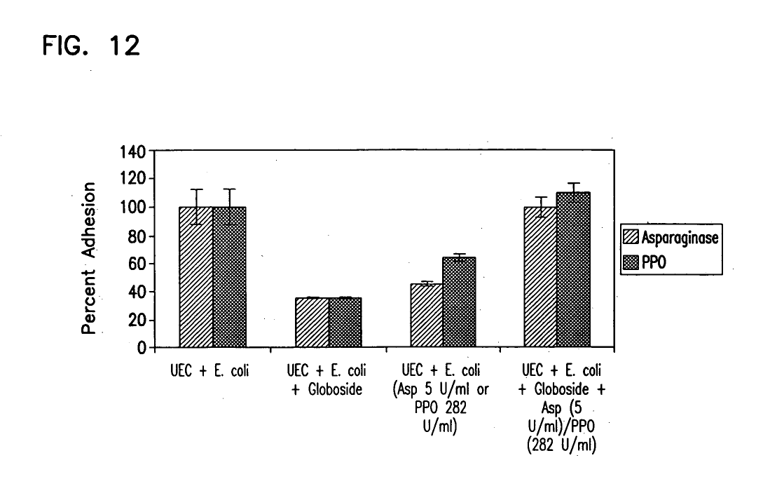

FIG. 13 illustrates inhibition by polyphenol oxidase of adhesion by S. pyogenes to buccal epithelial cells. Degree of adhesion is represented as a percentage based on the adhesion of untreated bacteria to UECs, which was set at 100%.

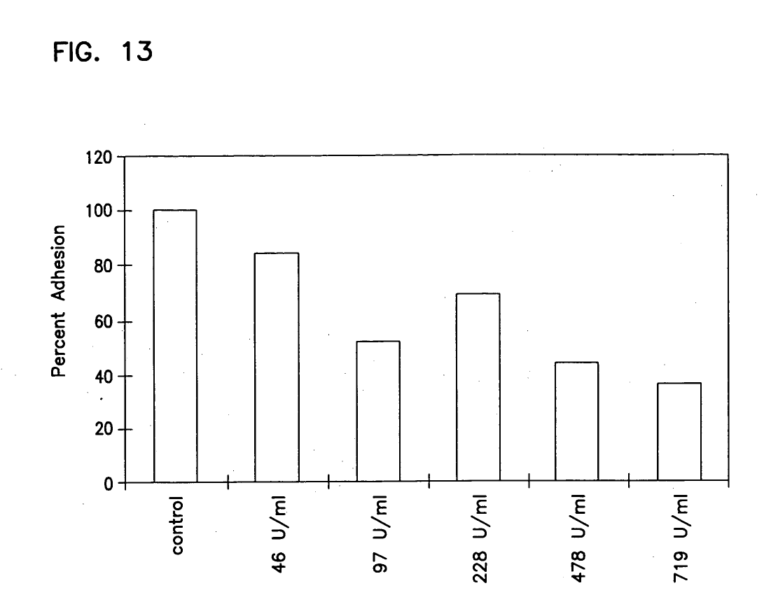

### DETAILED DESCRIPTION OF THE PREFERRED EMBODIMENTS

Definitions

As used herein, an enzyme that reduces or inhibits binding or adhesion of a microorganism to a cell, tissue, or surface includes any enzyme that when contacted with a cell reduces or inhibits binding or adhesion of that microorganism to a cell, tissue, or surface; preferably without killing or halting the growth of the microorganism. Such an enzyme preferably enzymatically modifies an adhesin on the microorganism, such as an adhesin with a binding site tyrosine and/or asparagine, a carbohydrate binding site, or another adhesin. For example, such an enzyme can catalyze a reaction for modifying a molecule on the microorganism. Such reactions include modification of a side chain of an amino acid. An enzyme employed in a method or composition of the invention can, for example, catalyze a reaction such as modification of an amino acid found in the binding site of an adhesin, such as a lectin or another carbohydrate binding site, on the microorganism. Preferably, the enzyme modifies a side chain of a residue important for binding by an adhesin molecule, such as an asparagine and/or tyrosine residue. Preferred enzymes that can be employed in the methods or compositions of the invention include polyphenol oxidase, asparaginase, or a combination thereof.

As used herein, “asparaginase” means an enzyme activity that catalyzes the hydrolysis of the side chain amide group of asparagine to a carboxyl group. That is, asparaginase catalyzes the conversion of an asparagine residue in a protein to an aspartate residue. A suitable asparaginase can be from any of a variety of organisms, and can be isolated from a natural source or produced recombinantly. Asparaginase includes enzymes identified by the E.C. number 3.5.1.1.

As used herein, “polyphenol oxidase” means an enzyme activity that catalyzes the oxidation of monophenols and/or ortho diphenols to ortho diquinones. Polyphenol oxidase, as used herein, includes enzymes known as catechol oxidase, monophenol monooxygenase, laccase, cresolase, tyrosinase, phenolase, catecholase, and phenol oxidase. The polyphenol oxidase includes enzymes assigned EC numbers 1.14.18.1 and 1.10.3.1. Polyphenol oxidases of the invention can be found in a variety of organisms including plants and fungi and are typically copper-containing oxidoreductases. A preferred polyphenol oxidase for use in the invention is found in, and isolated from, a thermophilic fungus, and more preferably, is produced recombinantly by expressing a vector containing a polyphenol oxidase gene in a host cell. Another preferred polyphenol oxidase for use in the invention is the polyphenol oxidase assigned EC number 1.14.18.1, also referred to in the art as polyphenol oxidase, monophenol monooxygenase, tyrosinase, phenolase, catecholase, or phenol oxidase.

Optional sources of polyphenol oxidase include mushrooms, plants, and thermophilic fungi. Suitable optional sources of polyphenol oxidase are described in the scientific literature including: Somkuti G. et al. Biotechnology Letters 15:773-778 (1993). Polyphenol oxidase can be produced either by isolation or purification from the natural source of the enzyme, or by recombinant expression of a vector or plasmid containing a polyphenol oxidase gene in a suitable host cell, and subsequent isolation or purification from the host cell.

Polyphenol oxidase has been isolated or purified from a variety of organisms, which is known to those of skill in the art. For example, polyphenol oxidase has been purified from Streptomyces michiganensis as reported by Phillips et al. J. Basic Microbiol. 31(4)293-300 (1991). Several polyphenol oxidase proteins have been sequenced and their structures characterized. These include enzymes from microbes such as Streptococcus thermophilus, Streptomyces glauscens, Neurospora crassa, and the like; and enzymes from plants such as broad bean, potato, tomato, and the like. See e.g., Somkuti et al. supra, Steffens et al. in Genetic Engr Plant Secondary Metabolism (Ellis B. E. et al. eds.) Plenum Press, NY, pp.275-312 (1994). Genes or mRNA encoding several polyphenol oxidases have been sequenced and expressed in host cells using standard methods. These include enzymes from plants such as broad bean and others. Polyphenol oxidase has been cloned and expressed in host cells using methods known to those of skill in the art. See, e.g., Robinson, S. P. et al. Plant Physiol. 99:317-323 (1992); and Lax, A. R. et al. in Enzymatic Browning and its Prevention, Amer. Chem. Soc., Washington D.C., pp. 120-128 (1995).

A preferred polyphenol oxidase for use in the invention has characteristics including one or more of the ability to catalyze oxidation of monophenols preferentially over diphenols; the ability to catalyze oxidation of one or more tyrosine residues of an adhesin molecule that are implicated in adhesion by a microorganism; and specificity for tyrosine containing substrata in preference to a polymer coating, a plastic, or a metal.

As used herein, “recombinant” enzyme means an enzyme that has been manipulated by a human at the DNA level. For example, the DNA encoding the enzyme can be expressed in a heterologous host cell. Alternatively, the DNA sequence encoding the naturally occurring enzyme is modified to produce a mutant DNA sequence which encodes the substitution, insertion, or deletion of one or more amino acids in the enzyme sequence compared to the naturally occurring enzyme.

As used herein, “microorganism” refers to microbes including a eukaryote, a prokaryote, or a virus, and including, but not limited to, a bacterium (either grain positive or gram negative), a fumgus, a virus, a protozoan, and other microbes or microscopic organisms.

As used herein, “adhesin molecule” or “adhesin” means a molecule or complex of molecules that is typically expressed on the surface of a microorganism and that mediates adhesion by the microorganism to cells, tissues, extracellular matrix, teeth, a dental prosthesis, a medical device or catheter, or another surface. Some adhesin molecules bind to a receptor on the surface of the other cell, tissue, or extracellular matrix. Some adhesin molecules adhere to polysaccharides that coat teeth, gums, dental prostheses, and the other tissues in the oral cavity. Some adhesin molecules adhere to polysaccharides or other molecules that coat body cavities, and tissues in these cavities, including the middle ear, vagina, and the like, or to other microorganisms that infect these cavities. Adhesins include carbohydrate binding proteins or sites on the surface of microorganisms, and adhesins with a binding site tyrosine residue and/or a binding site asparagine residue (which can be referred to tyrosine dependent adhesins, asparagine dependent adhesins, or tyrosine and asparagine dependent adhesins). Adhesin molecules include lectins, glucosyltransferases, lipoteichoic acids, hydrophobins, outer membrane proteins, flagella, fimbriae, pili, fibrillae, and the like.

As used herein, “binding site tyrosine residue” refers to a tyrosine residue of an adhesin molecule that is implicated in adhesion by a microorganism, such as by forming the binding site with which the adhesin molecule adheres. Such a tyrosine residue can be at, near, affecting, or important to this binding site. For example, a wide variety of bacterial adhesin lectins use tyrosine as a part of the carbohydrate binding site, either as part of the binding site itself and/or as part of the protein structure that maintains the shape of the binding site.

As used herein, “binding site asparagine residue” refers to an asparagine residue of an adhesin molecule that is implicated in adhesion by a microorganism, such as by forming the binding site with which the adhesin molecule adheres. Such an asparagine residue can be at, near, affecting, or important to this binding site. For example, a wide variety of bacterial adhesin lectins use asparagine as a part of the carbohydrate binding site, either as part of the binding site itself and/or as part of the protein structure that maintains the shape of the binding site.

As used herein, “adhesion by a microorganism” refers to the binding of a microorganism to a cell, tissue, extracellular matrix, a tooth, a dental prosthesis, or another surface, including hard surfaces that are cleaned by detergents or cleaners. The surface can be of a body cavity such as the oral cavity, vagina, middle ear, or the like.

As used herein, “reduce adhesion by a microorganism” or “reducing adhesion by a microorganism” refers to decreasing the amount of adhesion by the microorganism to a cell, tissue, extracellular matrix, a tooth, and/or dental prosthesis or to any other surface onto which microorganisms adhere and colonize. The decrease in adhesion can be observed by employing comparison to a control cell, tissue, extracellular matrix, a tooth and/or dental prosthesis, or to a control population. Generally, “reduce” or “reducing” can also be expressed as inhibit or inhibiting, diminish or diminishing, abolish or abolishing, and like terms. Reduction in adhesion by a microorganism by an amount that is measurable with statistical significance as less than a control value for adhesion by the microorganism can be expressed as “significantly reduced adhesion by a microorganism”. Significant reduction in adhesion by a microorganism can also be determined by demonstrating a desired biological effect upon treatment of a microorganism with enzyme, such as polyphenol oxidase and/or asparaginase, preferably including correlation of this effect with adhesion by the microorganism.

As used herein, “body cavity” refers to any cavity found in the body of an animal, such as the oral cavity, vagina, rectum, intestines, middle ear, nare (nostril), sinus, throat, esophagus, eustachian tube, bronchi, urinary bladder, urethra, and the like.

As used herein, “dental prosthesis” refers to a replacement for one or more of a mammal's teeth or another oral structure, including replacement of a single tooth, any type of denture, and any type of bridge. A dental prosthesis can be either fixed in the mammal's oral cavity or removable from the mammal's oral cavity. As used herein, “denture” refers to any type of denture including a partial denture, a complete denture, a fixed denture, and a removable denture.

As used herein “surface” refers to any surface to which a microorganism can bind or adhere. Surfaces include cells, tissues, extracellular matrix. Surfaces also include the surface of any catheter, implant, prosthesis, or other man made device that resides or is placed in or on a mammal's body or body cavity. Surfaces also include other surfaces to which a microorganism might bind such as a surface of a medical device external to the mammal, but that contacts the mammal or mammalian fluids or tissues, such as a periodontal dialysis apparatus, kidney dialysis apparatus, heart/lung machines, and the like. Surfaces also include surfaces in other apparatus or equipment to which microorganisms can adhere, such as in brewing apparatus, fermentation apparatus, effluent treatment apparatus, and other reactors and apparatus. Surfaces include hard surfaces that are cleaned by detergents or other cleaners.

As used herein, the terms “treating”, “treatment” and “therapy” refer to curative therapy, prophylactic therapy, and preventative therapy. Treating, treatment, and therapy can reduce or ameliorate the severity or presence of symptoms of a disorder, can reduce or ameliorate the severity or presence of a disorder, or can cure the disorder.

As used herein, the term “mammal” refers to any mammal classified as an animal, including humans, cows, horses, dogs and cats. In a preferred embodiment of the invention, the mammal is a human.

As used herein, the term “animal” refers to vertebrate animals including birds, mammals, reptiles, amphibians, and the like. Preferred animals include mammals and birds.

As used herein, the term “pharmaceutical composition” refers to a composition that can be administered to a subject, preferably a mammal, to treat a disorder that may benefit from administering an enzyme, such as polyphenol oxidase and/or asparaginase, to reduce adhesion by one or more microorganisms.

As used herein, the term “oral care composition” refers to a composition suitable for administration to the oral cavity of a subject, preferably a mammal, to treat a disorder of or in the oral cavity that may benefit from administering an enzyme, such as polyphenol oxidase and/or asparaginase, to reduce adhesion by one or more microorganisms.

As used herein, the term “effective amount” refers to an amount of an enzyme, such as polyphenol oxidase and/or asparaginase, sufficient to reduce or inhibit adhesion by a microorganism to a cell, tissue, extracellular matrix, a tooth, dental prosthesis or another surface, including hard surfaces that are cleaned by detergents or cleaners.

As used herein, “infection” refers to invasion and multiplication of one or more microorganisms in a tissue, cell, extracellular matrix, tooth, and/or dental prosthesis. Infection of a dental prosthesis refers to growth of the microorganism employing the prosthesis as a substratum, employing a biomolecule on the prosthesis as a substratum, or other mechanisms through which a microorganism can multiply in or on a dental prosthesis.

As used herein, “isolated,” when used to describe the various an enzyme, such as polyphenol oxidase and/or asparaginase, means an enzyme, such as polyphenol oxidase and/or asparaginase, that has been identified and separated and/or recovered from a component of its natural environment. Contaminant components of its natural environment are materials that can interfere with diagnostic or therapeutic uses for the enzyme, such as polyphenol oxidase and/or asparaginase, and may include enzymes, hormones, and other proteinaceous or non-proteinaceous solutes. Isolated an enzyme, such as polyphenol oxidase and/or asparaginase, includes an enzyme, such as polyphenol oxidase and/or asparaginase, in situ within host cells, since at least one component of the enzyme, such as polyphenol oxidase and/or asparaginase, natural environment will not be present. Ordinarily, however, isolated an enzyme, such as polyphenol oxidase and/or asparaginase, will be prepared by at least one purification step.

Methods and Compositions

The present invention includes methods and compositions employing an enzyme, such as polyphenol oxidase and/or asparaginase, for reducing adhesion by a microorganism; preferably without killing or halting the growth of the microorganism. The methods and compositions of the invention can reduce or inhibit binding or adhesion of a microorganism to a cell, tissue, or other surface. The methods and compositions of the invention employ any enzyme that when contacted with a cell reduces or inhibits binding or adhesion of that microorganism to a cell, tissue, or surface. The methods of the invention include administering effective amounts of the enzyme, e.g. polyphenol oxidase and/or asparaginase, for reducing adhesion by a microorganism, for example, at the site of an infection by a microorganism in an animal's body, including a body cavity, a dental prosthesis, a tissue, a site of catheterization, or the like. The compositions of the invention include effective amounts of an enzyme, such as polyphenol oxidase and/or asparaginase, in a carrier suitable for maintaining this enzyme in a form active for reducing adhesion by a microorganism. In one embodiment, the composition includes a pharmaceutical composition, suitable for therapeutic administration to an animal. The compositions of the invention also include oral, care compositions. The compositions of the invention also include compositions suitable for applying an enzyme such as polyphenol oxidase and/or asparaginase to a surface of a prosthesis, medical device (e.g. a catheter), a polymeric surface, a metal surface, or the like that can be cleaned with a cleaner or disinfectant.

The enzyme employed in the methods or compositions of the invention preferably enzymatically modifies an adhesin, such as a carbohydrate binding site, on the microorganism. Such an enzyme can catalyze a reaction for modifying an adhesin or other molecule on the microorganism or in the binding site of a lectin, or another carbohydrate binding site on the microorganism, e.g. modifying a side chain of an amino acid. Preferabl y, the enzyme modifies a side chain of an asparagine and/or tyrosine residue. Preferred enzymes that can be employed in the methods or compositions of the invention include a polyphenol oxidase, an asparaginase, or a combination thereof.

Adhesion by a microorganism can occur through a variety of mechanisms to a variety of substrata. For example, microorganisms that inhabit an animal's oral cavity can adhere to polysaccharides that coat teeth, gums, tongue, throat, cheeks, a dental prosthesis, and the other tissues in the oral cavity. Microorganisms also can adhere to cells, tissues, and extracellular matrix in or on the animal's body, or in or on one of the animal's body cavities. The cells can include microorganisms. Such microorganism to microorganism binding can be referred to as coaggregation. Microorganisms that coaggregate include microorganisms that are early and late colonizers of freshly cleaned teeth.

Microorganisms frequently employ adhesins, including carbohydrate binding sites, such as lectins and glucosyltransferases. Typical microorganismal lectins and glucosyltransferases, and other adhesins, can include one or more binding site tyrosine and/or asparagine residues. Modification of such tyrosine and/or asparagine residues can reduce binding by a microorganism to the polysaccharide or other substratum.

Some 200 carbohydrate-binding proteins have been analyzed in complex with their ligands, enabling detection of amino acid residues “in contact” (van der Waals contact, hydrogen bond contact, etc) with the carbohydrate. The Ligand-Protein Contact program available from the Protein Data Bank on the Research Collaborative for Structural Bioinformatics was employed to assess contact residues. For all classes of carbohydrate-binding proteins, the most frequent amino acid in contact with ligand was asparagine (Table A below). The third column of Table A illustrates that asparagine is over represented in the binding pocket, since its expected frequency in proteins is 5%. The vast majority of sites contain at least one asparagine, and one aromatic residue (Tyr, Trp or Phe) in contact with ligand. Among the aromatics, tyrosine appears to be the most common.

TABLE A
Amino Acids in Contact With Carbohydrate Ligands

| | | 2^{nd} Most | | | |
|---|---|---|---|---|---|
| | Most Frequent | Frequent | Asn as Percent | | |
| Binding | Contact Amino | Contact Amino | of Contact | % Sites With ≧ 1 | % Sites With ≧ 1 |
| Sites | Acid | Acid | Residues | Asn | Aromatic |
| Bacterial | Asn | Lys | 21 | 67 | 83 |
| (N = 24) | | | | | |
| Viral | Asn | Arg | 19 | 94 | 56 |
| (N = 18) | | | | | |
| Fungal | Asn | Tyr | 20 | 82 | 79 |
| (N = 28) | | | | | |
| Plant | Asn | Tyr | 12 | 73 | 73 |
| (N = 40) | | | | | |

Adhesin molecules that include binding site tyrosine residues include M-protein and fimbriae (London, J., Meth. Enzymol. 253:197-406 (1995)). The M-protein adhesin molecules have several tyrosine residues at their amino-terminus ends, which are believed to be binding site tyrosine residues (Cederval T. et al., Biochem. 36:4987-4994 (1997); Jones K. F. et al., J. Exp. Med. 164:1226-1238 (1986); Hasty D. L. et al., In: Fibronectin in Health and Disease. (S. E. Carsons, ed.), CRC Press, Boca Raton, pp.89-112 (1989)). Other bacterial adhesin molecules and surface proteins also include binding site tyrosine-residues (Comelissen, C. N. et al., Infect. Immun. 65:822-828 (1997); Lutwyche, P., R et al., Infect. Immun. 62:5020-5026 (1994); McNab, R., H. F. et al., Mol. Microbiol. 14:743-754 (1994); Nagata, H., A. et al., Infect. Immun. 65:422427 (1997); Sastry, A. et al., FEBS Lett. 151:253-256 (1983); Scalbert, A, Phytochem. 30:3875-3883 (1991); Schembri, M. A. et al. FEMS Microbiol. Lett. 137:257-63 (1996).) The adhesive subunit of type I fimbriae, called Fim H, has been crystallized and the binding interface described. Tyrosine and asparagine are both critical residues in the interaction with ligand (Choudhury et al., 1999, X-Ray Structure of the FimC-FimH Chaperone-Adhesin Complex from Uropathogenic Escherichia coli. Science 285:1061-1066). Influenza A virus possesses a lectin-like protein called hemagglutinin which recognizes terminal neuraminic acids on respiratory epithelial cells. Analysis of the 3D structure of the hemagglutinin complexed with its ligand showed that tyrosine and asparagine were both contact residues. Furthermore, these residues have been conserved in the virus strain since the last major antigenic shift in 1968, while residues around it have mutated frequently (Weis W. et al., Nature 333:426-431 (1988)). The adhesin molecules of E. histolytica also include binding site tyrosine residues. C. albicans has been reported to possess multiple adhesin molecules, such as hydrophobins and a lectin specific for GlcNAc.

Microorganisms that employ adhesin molecules having binding site tyrosine and/or asparagine residues include bacteria, such as Actinobacillus actinomycetemcomitans, Actinomyces israelii, A. naeslundii and A. viscosus, Capnocytophaga ochracea, Eikenella corrodens, Escherichia coli, Fusobacterium nucleatum, Haemophilus influenzae; Porphyromonas gingivalis, Prevotella intermedia, Proteus mirabilis, Proteus vulgaris, P. aeruginosa, P. loeschei, Streptococcus gordonii, S. mutans, S. oralis, S. sanguis, various group A streptococci, various invasive and antibiotic resistant staphylococci, and Treponema denticola; viruses such as influenza virus, specifically influenza A virus; yeasts, such as Candida albicans; and protozoans, such as Entamoeba histolytica. Adhesin molecules of several M5, M6 and M24 positive strains of streptococci have been studied (Dale J. B. et al., Vaccine 14-944-948 (1996); Courtney et al., REMS Microbiol. Letters 151:65-70 (1997)). P. aeruginosa makes a good model for study as its adhesion can depend on two lectins, PA-I and PA-2. Furthermore, the bacterium will form biofilms on a variety of surfaces, ranging from glass and steel to human lungs.

Numerous adhesin molecules include a binding site asparagine residue, including fimbriae, M-protein, and the like. Organisms having an adhesin molecules with a binding site asparagine residue include fungi, viruses, bacteria, and plants.

Adhesion by a microorganism can be determined by employing a variety of techniques known to those of skill in the art. These techniques include determining coaggregation of the microorganism of interest with a cell, such as through turbidimetry, determining aggregation of microorganisms with a polysaccharide, such as formation of an aggregate in solution or a pellicle, determining binding of the microorganism to a mammalian cell, monitoring hemagglutination, and determining binding of a microorganism to extracellular matrix. Hemagglutination has been used as a model to study adhesion by numerous bacteria to various tissues (Goldhar, J., Meth. Enzymol. 253:4349 (1995)). Typical oral pathogens, such as Eikenella corrodens, F. nucleatum, Haemophilus influenzae, P. gingivalis and T. denticola, have the ability to hemagglutinate human red cells (Leung K.-P. et al., Oral Microbiol. Immunol. 4:204-210 (1989); Nesbitt et al., Infect. Immun. 61:2011-2014 (1993); Socransky and Haffejee, J. Periodont. Res. 26:195-212 (1991); van Ham et al, J. Infect. Dis. 165:S97-99 (1992); Grenier, D., Oral Microbiol. Immunol. 6:246-249 (1991), and others). A microorganism treated with an enzyme, such as polyphenol oxidase and/or asparaginase, can be utilized to determine if the enzyme can affect measures of adhesion by the microorganism, such as hemagglutination titers, coaggregation of the microorganism, aggregation with the microorganism, or binding by the microorganism.

Typical assays for aggregation or coaggregation including microorganisms can be done in a suitable buffer and can involve visual end-point estimates or kinetic measurements, such as those employing a platelet aggregometer. Visual end-points can be determined by methods known to those of skill in the art, such as those described by Kolenbrander (Kolenbrander, P. E., Meth. Enzymol. 253:385-396 (1995)). In such a grading system O represents no aggregation; +1 represents small, evenly dispersed aggregates; +2 represents well-defined aggregates with some flocs; +3 represents large flocs with some background turbidity; and +4 represents a clear supernatant as result of massive flocculation. A platelet aggregometer works well when coaggregation reactions are reasonably rapid, such as completion within 15 min. The platelet aggregometer measures the disappearance of turbidity, and the results are continuously plotted on a strip chart recorder (Ofek and Doyle, supra). For non-aggregating pairs, the slope is zero. For others, the slope depends on nature and numbers of adhesin molecules and receptors.

Suitable reaction pairs of microorganisms for measuring coaggregation include:

| | | Control |
|---|---|---|
| | | Reaction can |
| | | Employ |
| Contains adhesin molecule: | Contains receptor: | Protection by: |
| A. viscosus T14V | S. oralis 34 | Lactose |
| Capnocytophaga ochracea | A. viscosus | Rhamnose |
| F. nucleatum | A. israelii | Lactose |
| F. nucleatum | A. actinomycetemcomitans | Unknown |
| Prevotella intermedia 27 | A. naeslundii | None |
| Prevotella loeschei | S. oralis | Lactose |
| S. gordonii | A. naeslundii | None |
| S. sanguis | Porphyromonas gingivalis | None |
| | W50 | |

Typically, in these pairs of microorganisms, the adhesin molecule is inactivated by heat and/or pronase, but the receptor is resistant to heat and/or pronase. These microbes represent early and late colonizers, Gram-positive and Gram-negative, those susceptible to protection by carbohydrates and those resistant to effects of carbohydrates. Additional coaggregating pairs been employed in studies reported in the Examples hereinbelow. Numerous other suitable coaggregating pairs are known to those of skill in the art and have been reported in the literature.

Adhesion of a microorganism to a cell from an animal tissue can be determined by any of a variety of methods known to those of skill in the art. Such methods include, for example, assaying E. coli strains possessing Pap-type fimbriae for adhesion to their substratum, di-galactose, by mixing them with suspensions of latex beads conjugated with the disaccharide (EY Laboratories) according to the procedure of Garcia et al. (Garcia E. et al., Curr. Microbiol. 17:333-337(1988)). For adhesion of the group A streptococci, human laryngeal cells (HEp-2 from ATCC) can be used as substrata. For such assays, bacteria can be tested at a variety of densities, starting, for example, at a high of 10⁹/ml with dilutions down to about 10⁷/ml or lower. These ranges can be used to generate a binding isotherm as described in Chapter 2 of Ofek and Doyle, 1994 supra. The adhesion reaction mixture can be incubated then aspirated and washed with medium to remove non-adherent, or adventitiously bound cells. The data obtained may yield, for example, regular binding isotherms, Langmuir plots, Scatchard plots and/or analysis of “cooperative” adhesion (Ofek and Doyle, supra). For example, if polyphenol oxidase abolishes a positive slope of a Scatchard plot of adhesion results, it could be said the enzyme is preventing positive cooperativity.

Adhesion of C. albicans to various substrata can be determined employing the general procedures of Hazen and Glee (Hazen, K. C. and Glee, P. M., Meth. Enzymol. 253:414-424 (1995)) and of Segal and Sandovsky-Losica (Segal, E. and Sandovsky-Losica, H., Meth. Enzymol. 253:439-452 (1995)). C. albicans for adhesion studies can be obtained from exponential (yeast phase) cultures in YE (yeast extract). Data can be treated as described above. In addition, plots of adhesion/buccal cell vs. numbers of cells added can be constructed in order to assess quantitative trends in enzyme mediated abolishment of adhesion function. C. albicans can infect denture wearers, head-neck irradiated patients, Sjögren's patients, AIDS patients, and other imrmunocompromised subjects.

A variety of microorganisms, such as oral bacteria, can adhere to many substrata including various extracellular matrix proteins. Adhesion to extracellular matrix proteins can be measured by a variety of methods known to those of skill in the art. For several oral bacteria, fibronectin is a receptor for their adhesin molecules. For others, collagen serves as a receptor. Other receptors are also known. (Ljungh, A. and Wadström, T. Meth Enzymol. 253:501-573 (1995)). Bacteria known to adhere to collagen include Actinomyces viscosus, Porphyromonas gingivalis, and Prevotella intermedia. (Liu, T., R. J. Gibbons, D. I. Hay, and Z. Skobe, Oral Microbiol Immunol. 6:1-5 (1991); Naito, Y., and R. J. Gibbons, J. Dent. Res. 67:1075-1080; Grenier, D., Microbiology 142:1537:1541 (1996)). Bacteria known to adhere to fibronectin binding include S. sanguis, S. pyogenes M5⁺ protein and Treponema denticola (reviewed in Ofek & Doyle, Bacterial Adhesion to Cells and Tissues. Chapman and Hall, New York. 1994). Adhesion to collagen can be studied by the method described by Grenier 1996 supra; and experiments with fibronectin can be patterned after those with collagen.

The microorganisms employed in studies of adhesion can be produced and isolated by any of a variety of methods known to those of skill in the art. For example, microorganisms can be purchased, obtained from clinical isolates, or prepared in other ways. Protozoa such as E. histolytica can be grown axenically as described by Petri and Schnaar (Petri, W. A. Jr. and Schnaar, R. L., Meth. Enzymol. 253:98-104 (1995)). The trophozoites can be harvested and washed as described. Adhesion studies can employ trophozoite membranes and hemagglutination to assay for the lectin. Viruses, such as influenza A virus, can be cultured, harvested, and handled according to procedures well known in the art. Virus particles can be treated with an enzyme such as asparaginase for various periods of time and at various concentrations, then assayed for hemagglutination by known methods, such as those reported by Casals, J. Meth. Virology III: 113-198 (1967)).

Administering an effective amount of an enzyme, such as polyphenol oxidase and/or asparaginase, to animal tissues, cells, extracellular matrix, teeth and/or dental prosthesis preferably results in a decrease in adhesion by one or more microorganisms sufficient to ameliorate detrimental effects or disease resulting from such adhesion. Effective administration or use of the enzyme, such as polyphenol oxidase and/or asparaginase, in this manner is typically evidenced by prevention or inhibition of infection, reduction or moderation of symptoms of an infection, reduction of adhesion, and the like. Absence or reduction of infection and moderation of symptoms can be determined by common clinical or laboratory methods. Reduction of adhesion can be determined by plate counts, ricroscopy, aggregometry, turbidimetry, isotopic labeling, and other methods standard in the art.

An enzyme, such as polyphenol oxidase and/or asparaginase, that decreases adhesion can be useful in one or more of a variety of applications including: fighting biofouling, for example in peritoneal dialysis; reducing dental caries; treating symptoms of infection by reducing adhesion of E. coli in an animal's gut; treating infection by reducing adhesion by one or more protozoa, such as Entamoeba; treating ulcers, for example by reducing adhesion of Helicobacter; treating viral infections by reducing adhesion of viruses, such as influenza virus; serving as a birth control agent; reducing contamination of eggs and/or other poultry products by serving as a chicken feed supplement for reducing levels in the bird of salmonellae; treating infection of periodontal tissue, eye, ear, or throat, such as by reducing adhesion by haemophilus, streptococcus, or candida; as a component of eye or ear drops, of a gargle (e.g. for sore throat), of a gels in a periodontal disease packing; killing mosquito larvae when cloned into Bt; as a probiotics (e.g. clone asparaginase into Lactobacillus); or fighting skin infections (impetigo) or Vibrio (which toxins bind CHO).

The methods and compositions of the present invention can be employed to treat urinary tract infections. Such infections are responsible for 9.6 million physician visits per year. The vast majority of these are caused by E. coli. Although nearly all E. coli strains express type 1 fimbriae, certain allelic variants of the fimbriae are associated with the ability to colonize the lower urinary tract. P-fimbriated E. coli are strongly associated with upper urinary tract (i.e., kidney) infections.

The methods and compositions of the present invention can be employed to treat infections at sites of catheters and/or cannulas. Organisms such as Proteus mirabilis, Proteus vulgaris and P. aeruginosa frequently colonize catheters, resulting in catheter removal and/or infection in the subject. Adhesion can lead to encrustation because the ammonia from urease will increase pH enough to precipitate struvite (Mg—NH₄-phosphate) and hydroxylapatite (Ca phosphate). It may be that asparaginase and polyphenol oxidase can inhibit urease, and/or adhesion. Or it may be that the enzymes reduce adhesion but not have any effects on urease. In either case, it is desired to reduce encrustation and prolong the life of the catheter. A model provided in some detail by Tunney et al. (1999, Biofilm and biofilm-related encrustation of urinary tract devices. Meth. Enzymol. 310:558-566) can be employed to demonstrate the effectiveness of an enzyme such as polyphenol oxidase and/or asparaginase against such adhesion related encrustation. Catheters and like instruments can be coated or otherwise treated with an enzyme, such as polyphenol oxidase and/or asparaginase to reduce or delay adhesion and/or encrustation.

The methods and compositions of the present invention can be employed to treat infections of body cavities, including the vagina and the middle ear. By treating infections of the vagina, the methods and compositions can also treat infections of newborns. Group B streptococcus is the most common cause of life threatening infections in newborns. The infection is acquired by infants during passage through the birth canal and also during the post-parturh period. Reducing adhesion of these microorganisms to the newborn or to the vagina can reduce or treat such infections. Streptococcus pneumoniae and Haemophilus influenzae are the #1 and #2 cause of middle ear infections (otitis media). Disrupting adhesion by these bacteria to epithelial or other cells of the ear can reduce or treat such infections.

The methods and compositions of the present invention can be employed to treat infections of nonhuman animals, such as birds, particularly chickens. Pharmaceutical compositions of the present inventions include compositions suitable for veterinary use. Treatment of animals used for meat or dairy products can be employed to prevent or reduce the incidence of food borne illnesses. For example, salmonellea contaminated eggs have been implicated more than any other source as causing food borne illness. Chicks that acquire S. enteritidis have the bacterium for life, leading to egg contamination. Disrupting adhesion by these bacteria to cells of the digestive or egg producing tracts of the chicks can treat such infections. An enzyme that reduces adhesion of a microorganism can be administered to the chick or other food producing animal in water, food, or by other suitable methods. Enzyme administered through food or water is preferably stable in the digestive tract, such as an enteric composition or a stabilized recombinant variant of the enzyme.

The methods and compositions of the present invention can also be employed against adhesion of microorganisms to synthetic surfaces, such as those of prostheses, of catheters or cannulas, of other medical devices or equipment, or of apparatus employed in brewing, fermentation, effluent treatment, and the like. Enzymes are commonly employed in cleaning or sanitizing compositions. Enzymes that reduce adhesion by microorganisms, such as polyphenol oxidase and/or asparaginase, can be formulated by methods and in formulations known to those of skill in the art for inclusion in cleaning and/or sanitizing compositions. In certain circumstances such enzymes can be employed during the process or treatment effected by the device or apparatus to reduce adhesion of microorganisms.

As shown in the examples below, contacting S. sobrinus with an enzyme, such as polyphenol oxidase and/or asparaginase, reduces adhesion by this microorganism. It is believed that this reduction in adhesion results from enzyme, such as polyphenol oxidase and/or asparaginase mediated inactivation of the glucan binding lectin of S. sobrinus. The observed reduction in adhesion had several manifestations. For example, an enzyme, such as polyphenol oxidase and/or asparaginase, reduces this bacterium's aggregation of soluble high-molecular weight dextran in a concentration-dependent and time-dependent manner. Mixing polyphenol oxidase with aggregates of S. sobrinus and dextran also reduces the reforming of aggregates. Polyphenol oxidase also reduced glucan synthesis by the S. sobrinus high-molecular weight glucosyltransferase isozyme.

Also as shown below, an enzyme, e.g. asparaginase and/or polyphenol oxidase, employed in the compositions and methods of the invention can, advantageously, reduce or inhibit adhesion or binding without killing or preventing growth of the microorganism. For example, polyphenol oxidase did not kill S. sobrinus, or type 1-fimbriated and P-fimbriated E. coli. Asparaginase did not kill or significantly inhibit growth of S. sobrinus, either of two strains of E. coli, S. pyogenes, Klebsiella pneumoniae, Bacillus cereus, or Proteus vulgaris.

As shown in the Examples below, contacting with an enzyme that modifies an adhesin molecule, such as polyphenol oxidase and/or asparaginase, can inhibit coaggregation of microorganisms such as periodontal pathogens implicated in periodontal infections and diseases. The periodontal pathogens include S. sanguis, Actinomyces naeslundii, Porphyromonas gingivalis, Actinobacillus actinomycetemcomitans, Fusobacterium nucleatum, Capnocytophaga ochracea, and Prevotella intermedia. Further, the trypsin-like protease activity is required for virulence of P. gingivalis. Polyphenol oxidase and asparaginase inhibit this protease.

As shown in the Examples below, contacting with an enzyme that modifies an adhesin molecule, such as polyphenol oxidase and/or asparaginase, can inhibit adhesion of a variety of microorganisms to a variety of substrata. The microorganisms include bacteria, such as E. coli, pneumococci, salmonellae (e.g. S. enteritidis), streptococci (e.g. S. pyogenes) and H. pylori.; viruses such as influenza virus; and amoeba, such as Entamoeba. The substrata include receptors, such as a mannose receptor; eukaryotic or mammalian cells, such as yeast, red blood cells, epithelial cells (e.g., urinary and buccal epithelial cells); matrix proteins, such as collagen or fibrin; and surfaces modeling biological surfaces, for example hydroxylapatite. Inhibition of adhesion by E. coli indicates that these enzymes can be employed in treating any of the variety of infections, diseases or disorders caused by or with symptoms from infection by E. coli. Inhibition of adhesion by H. pylori indicates that these enzymes can be employed in treating digestive tract ulcers. Inhibition of adhesion by influenza virus indicates that these enzymes can be employed in treating infection by influenza virus. Inhibition of adhesion by salmonellae indicates that these enzymes can be employed to treat or reduce the likelihood of infection by food borne microorganisms. Inhibition of adhesion by Entamoeba indicates that these enzymes can be employed to treat or reduce the likelihood of infection by amoeba. Inhibition of adhesion by yeast indicates that these enzymes can be employed to treat or reduce the likelihood of yeast infection. Inhibition of adhesion to extracellular matrix proteins indicates that these enzymes can be employed to treat or reduce disorders or symptoms caused by binding of microorganisms to extracellular matrix proteins, including invasion of tissues by microorganisms. The enzymes affect pathogenic (newly colonizing) bacteria disproportionately to long-term nonpathogenic colonizers (normal biota), which can facilitate therapeutic use of polyphenol oxidase or asparaginase.

Pharmaceutical Compositions

In one embodiment of the invention, there are provided pharmaceutical compositions including an enzyme, such as polyphenol oxidase and/or asparaginase. An enzyme, such as polyphenol oxidase and/or asparaginase, can be used in such pharmaceutical compositions, for example, for the treatment of microorganismal pathologies. It is contemplated that the pharmaceutical compositions of the present invention can be used to treat infections by one or more microorganisms that rely upon an adhesin molecule with a binding site tyrosine and/or asparagme residue.

The pharmaceutical compositions of the present invention preferably contain an effective amount of an enzyme, such as polyphenol oxidase and/or asparaginase, to reduce adhesion by a microorganism. The polyphenol oxidase is preferably a tyrosinase, a catecholase, a laccase, a peroxidase, or another oxidative enzymes acting on tyrosine residues. Optionally, the pharmaceutical composition may include agent(s) that stabilize or augment the activity of the polyphenol oxidase. Such agents include, but are not limited to, starch, gelatin, carrageenan, glycols andother agents used to compound pharmaceuticals.

The pharmaceutical compositions of the present invention include an enzyme, such as polyphenol oxidase and/or asparaginase, in a pharmaceutically acceptable carrier. Pharmaceutically acceptable carriers are known to those skilled in the art and include materials useful for the purpose of administering a medicament, which are preferably non-toxic, and can be solid, liquid, or gaseous materials, which are otherwise inert and medically acceptable and are compatible with the enzyme, such as polyphenol oxidase and/or asparaginase, and any other active ingredient that is present.

Water, saline, aqueous dextrose, and glycols are preferred liquid carriers, particularly (when isotonic) for injectable solutions. The carrier can be selected from various oils, including those of petroleum, mammal, vegetable or synthetic origin, for example, peanut oil, soybean oil, mineral oil, and sesame oil. Suitable pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, and ethanol. The compositions can be subjected to conventional pharmaceutical expedients, such as sterilization, and can contain conventional pharmaceutical additives, such as preservatives, stabilizing agents, wetting, or emulsifing agents, aerosolizing agents, salts for adjusting osmotic pressure, or buffers. Suitable pharmaceutical carriers and their formulations are described in Martin, “Remington's Pharmaceutical Sciences,” 15th Ed.; Mack Publishing Co., Easton (1975); see, e.g., pp. 1405-1412 and pp. 1461-1487. Such compositions will, in general, contain an effective amount of an enzyme, such as polyphenol oxidase and/or asparaginase, to reduce adhesion by a microorganism, together with a suitable amount of carrier so as to prepare the proper dosage form for proper administration to the animal.

The pharmaceutical compositions of the invention can be administered by various routes, including orally, used as a suppository or pessary; applied topically as an ointment, cream, aerosol, powder; or given as eye or nose drops, etc., depending on whether the preparation is used to treat internal or external infections by one or more microorganisms. The compositions can contain 0.1%-99% of the enzyme, such as polyphenol oxidase and/or asparaginase. Preferably, the composition includes about 0.1 wt-% to about 1.0 wt-% of an enzyme, such as polyphenol oxidase and/or asparaginase. The enzymes are usually soluble in pharmaceutical preparations.

For oral administration, fine powders or granules can contain diluting, dispersing and/or surface active agents, and can be presented in a draught, in water or in a syrup; in capsules or sacnets in the dry state or in a non-aqueous solution or suspension, wherein suspending agents can be included; in tablets or enteric coated pills, wherein binders and lubricants can be included; or in a suspension in water or a syrup. In some cases, the enzyme(s) may be formulated to form aerosols. Where desirable or necessary, flavoring, preserving, suspending, thickening, or emulsifying agents can be included. Tablets and granules are preferred, and these can be coated. A preferred formulation for oral administration includes agents that maintain the activity of an enzyme, such as polyphenol oxidase and/or asparaginase, in the stomach and intestines. Such agents include buffers and “slow release” components.

For buccal administration, the compositions can take the form of tablets or lozenges formulated in a conventional manner.

Alternatively, for infections of the skin or other external tissues the compositions are preferably applied to the infected part of the body of the animal as a topical ointment, cream or spray. The enzyme, such as polyphenol oxidase and/or asparaginase, can be presented in an ointment, for instance with a water-soluble ointment base, or in a cream, for instance with an oil in water cream base. Carriers for topical or gel-based forms of include polysaccharides such as methylcellulose, polyvinylpyrrolidone, polyacrylates, polyoxyethylene-polyoxypropylene-block polymers, polyethylene glycol, and wood wax alcohols. For topical administration, an enzyme, such as polyphenol oxidase and/or asparaginase, can be present in the pharmaceutical composition in a concentration of from about 0.01 to 10%, preferably 0.1 to 1.0% w/v. For topical administration, the daily dosage as employed for adult human treatment will range from 0.1 mg to 1000 mg, preferably 0.5 mg to 10 mg. However, it will be appreciated that extensive skin infections can require the use of higher doses.

For all administrations, conventional depot forms are suitably used. Such forms include, for example, microcapsules, nano-capsules, liposomes, plasters, inhalation forms, nose sprays, sublingual tablets, and sustained-release preparations. The enzyme, such as polyphenol oxidase and/or asparaginase, will typically be formulated in such carriers at a concentration of about 0.1 mg/ml to 100 mg/ml. An enzyme, such as polyphenol oxidase and/or asparaginase, cal) also be administered in the form of a sustained-release preparation. Suitable examples of sustained-release preparations include semipermeable matrices of solid hydrophobic polymers containing the protein, which matrices are in the form of shaped articles, e.g., films, or microcapsules. Examples of sustained-release matrices that are well known in the art include polyesters, hydrogels, polylactides, copolymers of L-glutamic acid and gamma ethyl-L-glutamate, non-degradable ethylene-vinyl acetate, degradable lactic acid-glycolic acid copolymers, and poly-D-(−)-3-hydroxybutyric acid.

An enzyme, such as polyphenol oxidase and/or asparaginase, can also be administered employing a composition suitable for gene therapy. For in vivo delivery of a nucleic acid (optionally contained in a vector) into an animal's cells, the nucleic acid is injected directly into the animal, usually at the sites where the polypeptide is required. Known in vivo nucleic acid transfer techniques include transfection with viral or non-viral vectors (such as adenovirus, lentivirus, Herpes simplex I virus, or adeno-associated virus (AAV)) and lipid-based systems (useful lipids for lipid-mediated transfer of the gene are, for example, DOTMA, DOPE, and DC-Chol; see, e.g., Tonkinson et al., Cancer Investigation, 14(1): 54-65 (1996)). A viral vector typically includes at least one element that controls gene expression, an element that acts as a translation initiation sequence, a packaging signal, long terminal repeats (LTRs) or portions thereof, and positive and negative strand primer binding sites appropriate to the virus used (if these are not already present in the viral vector). In addition, such vector typically includes a signal sequence for secretion of an enzyme, such as polyphenol oxidase and/or asparaginase, from a host cell in which it is produced.

Oral Care Compositions

The invention further provides oral care compositions including an enzyme, such as polyphenol oxidase and/or asparaginase. An enzyme, such as polyphenol oxidase and/or asparaginase, can be used in such oral care compositions, for example, for the treatment of pathologies in which a microorganism infects the oral cavity. It contemplates that the oral care compositions of the present invention can be used to treat any infection by one or more microorganisms that rely upon an adhesin molecule with a binding site tyrosine and/or asparagine residue.

The oral care compositions of the present invention preferably contain an effective amount of an enzyme, such as polyphenol oxidase and/or asparaginase, to reduce adhesion by a microorganism to cells or tissue of the oral cavity or to a dental prosthesis (e.g. a denture). The enzyme, such as polyphenol oxidase and/or asparaginase, is preferably resistant to Pasteurization, stable in compounding agents and amenable to formulation as a solid, liquid or aerosol. Optionally, the oral care composition may include agent(s) that stabilize or augment the activity of the enzyme, such as polyphenol oxidase and/or asparaginase. Such agents include trace metals, such as copper ions, and oxygen generating compounds, such as hydrogen peroxide.

Oral care compositions including an enzyme, such as polyphenol oxidase and/or asparaginase, can be used, for instance, for maintaining and/or improving oral hygiene in the oral cavity of mammals, and/or preventing or treating dental diseases in mammals. The present oral care compositions can also be used for reducing adhesion by one or more microorganisms to a dental prosthesis. For example, a denture can be cleaned with an enzyme, such as polyphenol oxidase and/or asparaginase, containing oral care composition either in the wearer's oral cavity or removed from the wearer's oral cavity. Oral care compositions of the invention include but are not limited to toothpaste, a dental cream, gel or tooth powder, a mouth wash or rinse, a denture cleaning agent (e.g. a cream or a soak), a chewing gum, a lozenge, and a candy. The oral care composition can be in the form of a solid, a semi-solid (e.g. a gel, a paste, or a viscid liquid), a liquid, or an aerosol.

Various ingredients that may be included in a tooth paste or gel and a mouth wash or rinse are well known in the art. In addition to an enzyme, such as polyphenol oxidase and/or asparaginase, a toothpaste or gel of the present invention will typically include one or more abrasives or polishing materials, foarning agents, flavoring agents, humectants, binders, thickeners, sweetening agents, or water. An enzyme, such as polyphenol oxidase and/or asparaginase, containing mouth wash or rinse will typically also include a water/alcohol solution and one or more flavors, humectants, sweeteners, foaming agents, and colorants.

Suitable, known abrasives or polishing materials include alumina and hydrates thereof (e.g. alpha alumina trihydrate), magnesium trisilicate, magnesium carbonate, sodium bicarbonate, kaolin, aluminosilicates (e.g. aluminum silicate), calcium carbonate, zirconium silicate, powdered plastics (e.g. powdered polyvinyl chloride, polyamide, or various resins) xerogels, hydrogels, aerogels, calcium pyrophosphate, water-insoluble alkali metaphosphates, dicalcium phosphate and/or its dihydrate, dicalcium orthophosphate, tricalcium phosphate, particulate hydroxylapatite, and mixtures of these abrasives or polishing materials. Typically, the abrasive or polishing material can be present in from 0 to about 75% by weight, preferably from 1% to about 65%, more preferably, for toothpastes or gels, about 10% to about 55% by weight of the toothpaste or gel.

Suitable, known humectants, which are typically employed to prevent loss of water from a toothpaste or gel, or other composition, include glycerol, polyol, sorbitol, polyethylene glycols (PEG), propylene glycol, 1,3-propanediol, 1,4-butane-diol, hydrogenated partially hydrolyzed polysaccharides, and mixtures of these humectants. In a toothpaste or gel, humectants are typically at about 0% to about 75%, preferably about 5 to about 55% by weight of the composition.

Suitable, known thickeners and binders, which maintain stability of an oral care composition include silica, starch, tragacanth gum, xanthan gum, extracts of Irish moss, alginates, pectin, certain cellulose derivatives (e.g. hydroxyethyl cellulose, carboxymethyl cellulose, or hydroxy-propyl cellulose), polyacrylic acid and its salts, and polyvinyl-pyrrolidone. Typically, a toothpaste or gel includes about 0.1% to about 20% by weight of one or more thickeners and about 0.01% to about 10% by weight of one or more binders.

A suitable foaming agent or surfactant in such oral care compositions will typically not significantly decrease the activity of an enzyme, such as polyphenol oxidase and/or asparaginase, present in the composition. Such foaming agents or surfactants can be selected from anionic, cationic, non-ionic, and amphoteric and/or zwitterionic surfactants. These can include fatty alcohol sulphates, salts of sulphonated mono-glycerides or fatty acids having 10 to 20 carbon atoms, fatty acid-albumin condensation compositions, salts of fatty acids amides, taurines, and/or salts of fatty acid esters of isothionic acid. The foaming agent or surfactant can be at levels in the composition from about 0% to about 15%, preferably from about 0.1% to about 10%, more preferably from 0.25 to 7% by weight.

Suitable, known sweeteners include artificial sweeteners such as saccharin and aspartame. Suitable, known flavors include spearmint and peppermint. Such flavors or sweeteners are typically present at levels from about 0.01% to about 5% by weight, or from about 0.1% to about 5%.

The oral care compositions of the invention can also include one or more added antibacterials, anti-calculus agents, anti-plaque agents, compounds which can be used as fluoride source, dyes/colorants, preservatives, vitamins, pH-adjusting agents, anti-caries agents, or desensitizing agents.

An oral care composition including an enzyme, such as polyphenol oxidase and/or asparaginase, can be applied to the oral cavity of a mammal employing any of numerous methods known in the art for administering oral care compositions. For example, the oral care composition can be applied as any commonly applied toothpaste or mouthwash. The oral care composition can be introduced into the oral cavity, applied to an oral tissue, such as teeth and/or gums, removed from the oral cavity (e.g. by rinsing), and the oral cavity can be rinsed. Alternatively, the oral care composition can be applied to the periodontal pocket as a semi-solid or as a solid implant. A gel, paste, or viscid liquid can be applied with, for example, a toothbrush, a swab, a finger, a syringe, or a dentist's tool. In yet another embodiment, the oral care composition can used to soak a denture. For example, the denture can be removed from the oral cavity of the wearer and immersed in a solution or suspension including an enzyme, such as polyphenol oxidase and/or asparaginase. Another embodiment involves the use of aerosols to administer effective doses.

Oral care compositions can be made using methods known in the art for making oral care compositions. The oral care compositions can contain 0.1%-99% of the enzyme, such as polyphenol oxidase and/or asparaginase. Preferably, the composition includes about 0.1 wt-% to about 1.0 wt-% of an enzyme, such as polyphenol oxidase and/or asparaginase.

Yet another embodiment of the composition of the invention includes a composition suitable for reducing or inhibiting adhesion or binding of microorganisms to hard surfaces, including dental prostheses, medical devices, implants, counters, porcelain or plastic fixtures, instruments, and the like. Such a composition can be a cleaner or detergent composition including an enzyme such as polyphenol oxidase and/or asparaginase. Formulations for cleaners or detergents that are will not inactivate, or that will support activity of, enzymes such as polyphenol oxidase and asparaginase are known to those of skill in the art.

Articles of Manufacture

The invention further provides articles of manufacture. An article of manufacture such as a kit containing an enzyme, such as polyphenol oxidase and/or asparaginase, useful for reducing adhesion by a microorganism, or for the treatment of the disorders described herein, includes at least a container and a label. Suitable containers include, for example, bottles, vials, syringes, and test tubes. The containers may be formed from a variety of materials such as glass or plastic. The container holds a composition that is effective for treating the condition and may have a sterile access port. The active agent in the composition is the enzyme, such as polyphenol oxidase and/or asparaginase. The label on, or associated with, the container indicates that the composition is used for treating the condition of choice. The article of manufacture may further include a second container including a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use. The article of manufacture may also include a second or third container with another active agent as described above.

The present invention may be better understood with reference to the following examples. These examples are intended to be representative of specific embodiments of the invention, and are not intended as limiting the scope of the invention.

### EXAMPLES

### Example 1

### Inhibition of Aggregation of Streptococcus sobrinus by Polyphenol Oxidase and by Asparaginase

Polysaccharides provide an important substratum for adhesion and aggregation of microorganisms. Studies of the effect of polyphenol oxidase and/or asparaginase on adhesion by S. sobrinus to dextran demonstrated that treatment with either of these enzymes reduces adhesion by microorganisms to polysaccharides.

Materials and Methods

Bacteria and Growth Conditions

S. sobrinus 6715 was maintained on tryptic soy agar (Difco, Detroit, Mich.) and grown for experiments in Terleckyj's defined medium or tryptic soy broth (TSB) (Difco) pretreated with dextranase (1 mg of enzyme per g dry medium) and invertase (5 mg per g dry medium) for 2 h each in 5% CO₂ at 37° C. for 16-18 h. Cultures were centrifuged at 12,000×g for 10 min and washed twice with cold phosphate buffered saline (PBS, 20 mM, pH 7.2). Cells were suspended in PBS to an optical density of 0.8 (A₅₄₀). Cultures grown in TSB were harvested and suspended in PBS and subsequently treated with dextranase for 1 h, followed by washing two times in PBS.

Aggregation Assays

The standard rate assay published by Drake et al. (Drake D. et al. Infect. Immun. 56:1864-1872 (1988)) was used to study the interaction of S. sobrinus 6715 glucan-binding lectin with high molecular weight dextran. Briefly, bacterial suspensions were adjusted to an optical density of 0.75-0.90 in PBS, and 3-ml suspensions were added to test tubes (13×100 mm). Dextran T-2000 was added at a final concentration of 10 μg/ml and the suspensions were vortexed for 5 sec. Control tubes received PBS. The decrease in optical density was continuously monitored spectrophotometrically for 5 min. Rate constants were obtained from the slopes of first-order plots of lnA/Aₒ (A=observed optical density; Aₒ optical density at time zero) versus time in minutes. Each sample was assayed at least three times.

Aggregation Competition Assays

The carbohydrates dextran T-10 (molecular weight 10,000, 1 mg/ml) or glycogen (2.5 mg/ml, Sigma) were added to the aggregation assays before addition of the high molecular-weight dextran.

Reducing Aggregation with Enzyme

Cells were suspended in phosphate buffer (20 mM, pH 6.5) and treated with either polyphenoi oxidase (Worthington Biochemical Corporation, Freehold N.J., Lot 37A889) (1260 U/ml) for 1 h at 37° C. with rotary shaking (150 rev/min), then washed twice with cold PBS. Inhibitors (all from Sigma), when present, were added before polyphenol oxidase addition at the following concentrations: phenylmethylsulfonyl fluoride (PMSF) 500 μM; leupeptin, 500 μg/ml; ethylenediaminetetracetic acid (EDTA), 5 mM, potassium chloride 200 mM; polyvinylpyrrolidone, 500 μg/m; ascorbic acid, 3 mM; lactic acid, 10%.

In another experiment cells were treated with asparaginase.

Results

Reducing Glucan Aggregation with Polyphenol Oxidase

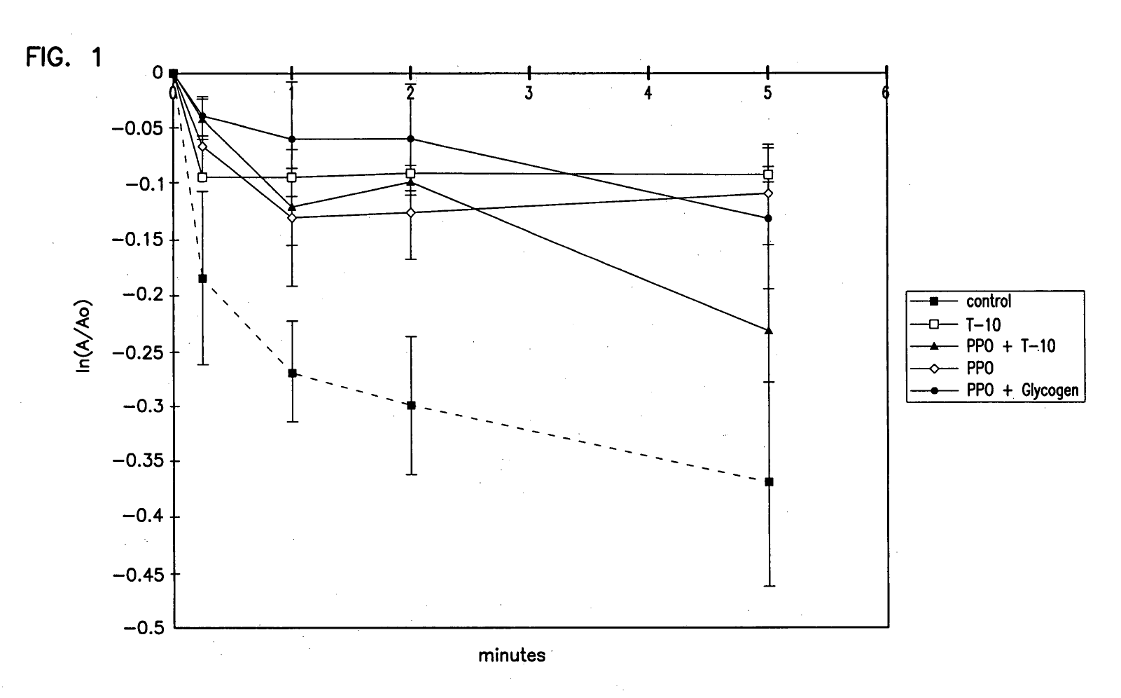

FIG. 1 depicts the decrease in absorption accompanying S. sobrinus 6715 glucan binding lectin complexing with high molecular weight dextran when growth took place in complex medium (tryptic soy broth). Cells grown in the Terleckyj defined medium required seven-fold lower concentrations of polyphenol oxidase for inhibition (data not shown).

Polyphenol oxidase reduced aggregate formation to approximately the level seen when low-molecular weight glucan (dextran T-10) was included in the reaction (FIG. 1). Pre-incubating the bacteria with T-10 before polyphenol oxidase treatment resulted in significant blocking of the polyphenol oxidase inhibition of aggregation. Glycogen pre-incubation, in contrast, had no effect. Protease inhibitors PMSF and leupeptin completely inhibited polyphenol oxidase induced inactivation.

Known polyphenol oxidase inhibitors, such as EDTA, ascorbic acid, polyvinylpyrrolidone, lactic acid and increased Cl⁻ ion, reduced the effects of polyphenol oxidase on glucan aggregation. Similarly, when polyphenol oxidase activity was reduced by lowering the temperature, the glucan binding lectin activity was not appreciably altered (Table 1).

TABLE 1
The Effect of Inhibiting Polyphenol Oxidase
Activity on Dextran Aggregation

| | | | Percent Inhibition of |
|---|---|---|---|
| Inhibitor of Polyphenol | | | Polyphenol Oxidase Induced |
| Oxidase | Concentration | | Reduction in Aggregation |
| EDTA | 3 | mM | 100 |
| ascorbic acid | 3 | mM | 100 |
| polyvinylpyrrolidone | 500 | μg/ml | 100 |
| lactic acid | 10% | | 92 (+/−8) |
| KCl | 2.5% | | 100 |
| 4° C. | — | | 51 (+/−31) |
| PMSF | 500 | μM | 0 |
| leupeptin | 500 | μg/ml | 0 |

Reversal of Aggregation by Polyphenol Oxidase

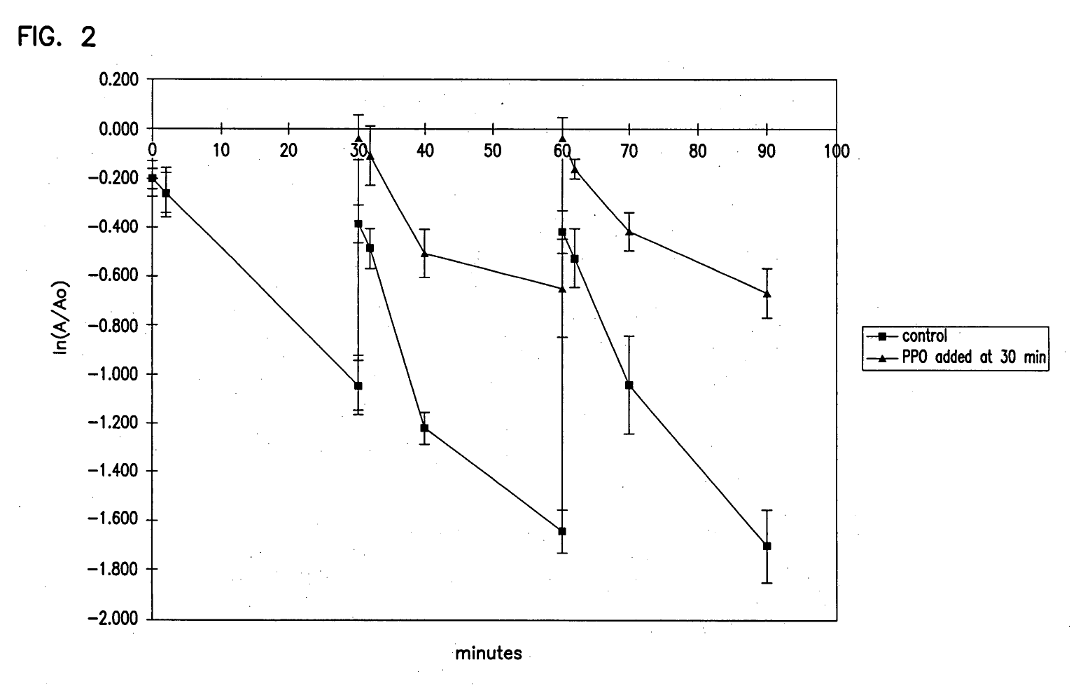

When polyphenol oxidase was mixed with pre-formed glucan binding lectin-glucan aggregates, aggregate reformation was significantly retarded. This phenomenon was repeated after remixing the suspension with no further polyphenol oxidase addition (FIG. 2).

Reduction of Pellicle Formation by S. sobrinus Cell-Bound Glucosyltransferase

Table 2 shows that 1 mg/ml polyphenol oxidase effectively inhibited the formation of pellicle formed by cultures of S. sobrinus grown in the presence of sucrose.

TABLE 2
Aggregation Scores for Inhibition of Cell-Bound
Glucosyltransferase by Polyphenol Oxidase

| | no sucrose | sucrose (200 mM) |
|---|---|---|
| no polyphenol oxidase | — | ++++^{a} |
| polyphenol oxidase (1 mg/ml) | — | + |

^{a}control tubes with no bacteria showed no aggregation

Discussion and Conclusions

Polyphenol oxidase is a tetrameric metalloenzyme, requiring four copper ions per enzyme molecule. (Vamos-Vigyazo, L., Crit. Rev. Food Sci. Nutr. 15:49-127 (1981)). Therefore, certain metal chelating agents have been found to be inhibitory to its activity. (Walker, J. R. L., Enzyme TechnoL Dig. 4:89 (1975)). Inhibition of the effects of polyphenol oxidase by EDTA (Table 1) supports this finding. In this study, the microorganismal glucan binding lectin binding activity was significantly reduced by pretreatment with polyphenol oxidase. The effect of polyphenol oxidase was masked when cells grown in nutrient medium were not first treated with dextranase. This suggests that low-molecular weight dextrans manufactured by cell-bound glucosyltransferase from trace sugars in the medium were tightly bound to the glucan binding lectin binding site. This effect mimics the blocking experiments conducted by pre-incubating the reaction mixture with dextran T-10. Cells grown in sucrose-free defined medium needed no dextranase treatment for polyphenol oxidase to be effective.

The binding constant for high molecular weight dextrans appears to be lower than for low-molecular weight dextrans, since either gentle vortexing or the presence of polyphenol oxidase could apparently displace dextran T-2000 (FIG. 2), but not T-10, from glucan binding lectin.

Reducing Glucan Aggregation with Asparaginase

Treatment of S. sobrinus with asparaginase reduced the ability these cells to aggregate high molecular weight dextrans.

### Example 2

### Inhibition of Streptococcus sobrinus Glucosyltransferase by Polyphenol Oxidase or by Asparaginase

Some microorganisms employ an alternate binding site on glucosyltransferase for adhesion to other cells and tissues. The effect of polyphenol oxidase or asparaginase on glucosyltransferase was studied to demonstrate a mechanism by which polyphenol oxidase or asparaginase can reduce adhesion by a microorganism.

Materials and Methods

Cells were grown and certain procedures conducted as described in Example 1. Additional procedures were conducted as follows.

Inhibition of Cell-Bound Glucosyltransferase

S. sobrinus 6715 was inoculated into tubes (5 ml) of TSB. Some tubes contained additionally either sucrose (final concentration=200 mM) and/or polyphenol oxidase (final concentration=1.0 mg/ml). After 18 h of growth, tubes were examined for formation of pellicle on the glass surface.

Inhibition of Purified Glucosyltransferase-I and Glucosyltransferase-S

Glucosyltransferase activity was detected as described in the art, with some modifications described hereinbelow. Protein samples were mixed with electrophoresis sample buffer and incubated for 1 to 4 h at 37° C. before the gel was run. Nonfixed gels were incubated in 50 mM sodium acetate (pH 5.5) containing 1% vol/vol Triton X-100, 2% (wt/vol) sucrose, and 0.07% (wt/vol) NaN₃ at 37° C. for 24 h. Gels were also incubated in the same buffer with glucan T2000 (2 to 4 mg/ml) or fluorescein isothiocyanate-conjugated glucan T-10 (2 mg/ml) to detect both glucosyltransferases and glucan binding proteins (GBPs). After incubation, the gels were fixed for 30 min in 75% ethanol and rocked on a shaker for 30 min with 0.7% periodic acid in 5% acetic acid. The gels were then shaken for 1 h in 0.2% (wt/vol.) sodium metabisulfite in 5% acetic acid. After two additional treatments in sodium metabisulfate and acetic acid, the gels were placed in Schiffs reagent for 0.5 to 1 h. Finally the gels were washed extensively in 45% methanol-45% acetic acid-10% H₂O for destaining. In some experiments, purified protein was treated with 100 μg/ml polyphenol oxidase for 1 h, or with asparaginase, before loading on the gel.

Results, Discussion, and Conclusions

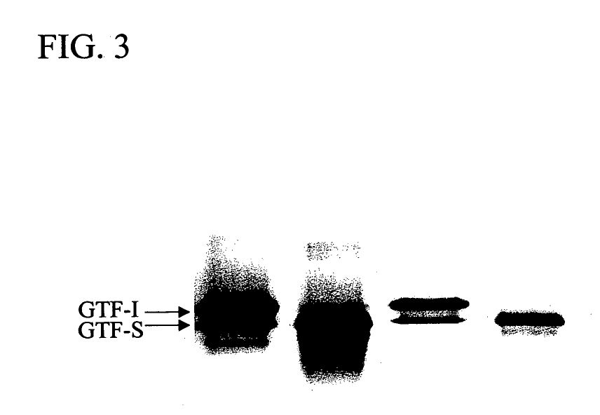

The higher molecular weight glucosyltransferase-I (approxim ately 145 kda) was unable to produce dextrans from sucrose after pre-incubation with 100 μg/ml polyphenol oxidase (FIG. 3), as evidenced by species remaining unstained by Schiff s reagent. An activity gel (activity on hydrolysis of sucrose following renaturation of an SDS-PAGE gel) revealed a loss of activity (not shown). Similarly, an enhanced chemiluminescence gel revealed the loss of glucan binding (not shown). No inhibition of glucosyltransferase-S (approximately 135 kda) was observed.

Glucosyltransferase, the streptococcal enzyme responsible for synthesizing glucans from dietary sucrose, also has glucan-binding activity which is spatially distinct from its sucrose binding site. (Mooser, G. and C. Wong, Infect. Immun. 56:880-884 (1988)). As shown herein, polyphenol oxidase effectively reduced glucan manufacture, seen as pellicle formation in growing cultures (Table 2). This reduction appears to be due to inhibition of glucosyltransferase-I by the polyphenol oxidase (FIG. 3). The combination of glucan binding lectin- and glucosyltransferase-I inhibition may therefore have effects on colonization of teeth by S. sobrinus.

Polyphenol oxidase was shown to inhibit the glucan-binding activity of glucosyltransferase. Polyphenol oxidase also prevented glucan manufacture by glucosyltransferase. Such inhibition may provide a mechanism through which polyphenol oxidase reduces adhesion by a microorganism.

Inhibition of Purified Glucosyltransferases by Asparaginase

Treatment of purified S. sobrinus glucosyltransferase with asparaginase reduced binding by the glucosyltransferase to high molecular weight dextrans. An activity gel (activity on hydrolysis of sucrose following renaturation of an SDS-PAGE gel) revealed a loss of activity (not shown). Similarly, an enhanced chemiluminescence gel revealed the loss of glucan binding (not shown).

### Example 3

### Neither Polyphenol Oxidase nor Asparaginase Kill Microbes

To eliminate lethality as a possible cause of the inhibitory effects of polyphenol oxidase or asparaginase, these enzymes were evaluated for their ability to kill several types of microbes, typically bacteria. Neither enzyme killed any microbes tested.

Materials and Methods

Bacteria were grown and polyphenol oxidase and asparaginase were used according to methods described in the previous and following Examples, with the exceptions below.

Effect of Polyphenol Oxidase on Growth of S. sobrinus

Standard disc-diffusion assays were performed with 500 μg/ml and 1.0 mg/ml of polyphenol oxidase. Duplicate series of ten-fold dilutions (to 10⁻⁸) of late exponential phase cultures of S. sobrinus were made in PBS. Eighty μl of each concentration of polyphenol oxidase was pipetted onto 13 mm sterile filter paper discs. The discs were applied to the center of agar plates previously inoculated with lawns of S. sobrinus. Plates were incubated for 18 h and examined visually for any effect of polyphenol oxidase on growth of the bacteria.

Effect of Asparaginase on Growth of S. sobrinus

Possible inhibition of growth of S. sobrinus was typically determined as follows: Standard disc-diffiusion assays were performed with 500 μg/ml and 1.0 mg/ml of asparaginase. Duplicate series of ten-fold dilutions (to 10′) of late exponential phase cultures of S. sobrinus were made in PBS. Eighty μl of each concentration of asparaginase was pipetted onto 13 mm sterile filter paper discs. The discs were applied to the center of agar plates previously inoculated with lawns of S. sobrinus. Plates were incubated for 18 h and examined visually for any effect of asparaginase on growth of the bacteria.

Effects of Asparaginase or Polyphenol Oxidase on Other Microbes

Similar and/or art recognized methods were used to monitor the effects of asparaginase on growth of other microbes including two strains of E. coli, S. pyogenes, Klebsiella pneumoniae, Bacillus cereus, and Proteus vulgaris. Both type 1-fimbriated and P-fimbriated E. coli were incubated with polyphenol oxidase, asparaginase, and ampicillin to test the ability of these agents to inhibit the growth of the bacteria.

Results

No inhibition of growth of S. sobrinus 6715 was seen in the disc-diffusion assays using discs impregnated with polyphenol oxidase. Asparaginase did not significantly inhibit growth of S. sobrinus, either of two strains of E. coli, S. pyogenes, Klebsiella pneumoniae, Bacillus cereus, or Proteus vulgaris.

All concentrations of ampicillin (1 mg/ml to 0.015 mg/ml) inhibited the growth of each of type 1-fimbriated and P-fimbriated E. coli. Neither polyphenol oxidase (1128 units/ml to 17.5 units/ml) or asparaginase (20 units/ml to 0.31 units/ml) concentrations tested were able to inhibit the growth of type 1-fimbriated and P-fimbriated E. coli (data not shown).

### Example 4

### Inhibition of Adhesion of Periodontopathogens by Polyphenol Oxidase and Asparaginase

To aid in evaluating their usefulness against periodontal infections and diseases, polyphenol oxidase and asparaginase were studied and determined to inhibit adhesion by several periodontal pathogens.

Materials and Methods

All bacteria were purchased from the American Type Culture Collection and included S. sanguis 10556, Actinomyces naeslundii strains T14V and 12104, Porphyromanas gingivalis W50, Actinobacillus actinomycetemcomitans 33384, Fusobacterium nucleatum 25586, Capnocytophaga ochracea 27872 and Prevotella intermedia 25611. A type I fimbriated Escherichia coli was provided by Prof. D. L. Hasty, VAMC,. Memphis, Tenn. S. sanguis 10556, A. naeslundli strains 12104 and T14 and E. coli were grown static cultures in Todd-Hewitt broth (BBL Microbiology Systems, Cockeysville, Md.). P. gingivalis W50, A. actinomycetemcomitans and C. ochracea were grown in brain heart infusion broth (BBL). F. nucleatum 22586 and P. intermedia 25611 were grown in modified Schaedler broth (BBL). All oral species except S. sanguis 10556 were incubated at 37° C. as static cultures under an anaerobic atmosphere containing H2, CO₂ and N₂ (10:10:80) with GasPaks (BBL). S. sanguis was incubated at 37° C. as static cultures in 5% CO₂.

Bacteria were harvested in the mid to late exponential growth phase by centrifugation (10,000×g for 10 min at 4 CC). The cells were washed twice in coaggregation buffer (30 mM 3-[N-morpholino]propanesulfonic acid (MOPS), pH 7.0) and adjusted to an optical density of 0.6-0.8 at 540 nm (1-cm path). Final volume of 3 ml were employed containing both coaggregating partners. Cells were incubated 60 min at 37° C., washed 2×in phosphate buffer (PB), suspended to an OD of 0.8 and mixed with their partners. Adhesins on cells could be inactivated by heating for 15 min at 100° C. Enzyme concentrations were 20 U/ml for asparaginase and 90 U/ml for polyphenol oxidase polyphenol oxidase, mushroom polyphenol oxidase; Per, horseradish peroxidase; asparaginase, E. coli asparaginase.

Coaggregation was assessed by turbidity changes. Following mixing of the cells for 10 s on a Vortex mixer, absorbance readings were made every 30 min. The percentage of coaggregation was calculated from the following formula: Percent coaggregation [OD₅₄₀(A)+OD₅₄₀(B)-OD₅₄₀(A+B)]/(OD₅₄₀(A)+OD₅₄₀(B)), where A is the control containing cells of one strain, B is the control containing cells of the other strain, and A+B is the reaction mixture containing both cells. The coaggregation assays were repeated three times to confirm the reproducibility of the data.

The hemagglutinations were carried out in 96 microwell (Nunc) plates in which 50 μl of 1% suspension of sheep blood and 50 μl of serially diluted cells suspension in phosphate buffered (10 mM Na₂HPO₄, 10 mM KH₂PO₄, 150 mM NaCl, 3 mM KCl) were added. The cells were adjusted at 1.4 OD at 540 nm. All the strains were treated in NaCl-free buffer with polyphenol oxidase (90 U/ml) or asparaginase (20 U/ml) for 90 min at 37° C. before hemagglutination.

Results

Results are shown in Tables 3 and 4. Table 4 shows results of hemagglutination assays involving periodontopathogens.

TABLE 3
Coaggregation of Periodontopathogens Following Enzymatic Treatments

| Adhesin | Receptor | Inhibition (%) |
|---|---|---|
| B. fragilis (polyphenol oxidase) | A. naeslundii ATCC 12104 | 41 |
| B. fragilis (Per) | A. naeslundii ATCC 12104 | 11 |
| P. gingivalis W50 (polyphenol oxidase) | A. naeslundii T14V | 37 |
| P. gingivalis W50 | A. naeslundii T14V (polyphenol oxidase) | Enhanced |
| P. gingivalis W50 (asparaginase) | A. naeslundii T14V | 31 |
| A. naeslundii ATCC 12104 (polyphenol oxidase) | Capnocytophaga ochracea | 19 |
| A. naeslundii ATCC 12104 (asparaginase) | Capnocytophaga ochracea | 25 |
| S. sanguis 10556 | A. naeslundii ATCC 12104 (polyphenol oxidase) | 29 |
| A. naeslundii ATCC 12104 (polyphenol oxidase) | P. intermedia | 15 |
| A. naeslundii ATCC 12104 | P. intermedia (polyphenol oxidase) | 27 |
| A. naeslundii ATCC 12104 | P. intermedia (asparaginase) | 23 |
| F. nucleatum | A. actinomycetemcomitans (polyphenol oxidase) | 27 |
| F. nucleatum | A. actinomycetemcomitans (asparaginase) | 40 |
| F. nucleatum (polyphenol oxidase) | A. actinomycetemcomitans | 17 |
| F. nucleatum (asparaginase) | A. actinomycetemcomitans | 23 |

TABLE 4
Hemagglutination assays of periodontopathogens

| Dilutions | 1/2 | 1/8 | 1/16 | 1/32 | 1/64 | 1/128 | 1/256 | 1/512 |
|---|---|---|---|---|---|---|---|---|
| P. gingivalis | + | + | − | − | − | − | − | − |
| P. gingivalis (polyphenol oxidase) | − | − | − | − | − | − | − | − |
| P. gingivalis (asparaginase) | − | − | − | − | − | − | − | − |
| C. ochracea | − | − | − | − | − | − | − | − |
| C. ochracea (polyphenol oxidase) | + | + | − | − | − | − | − | − |
| P. intermedia | + | + | + | + | + | − | − | − |
| P. intermedia (polyphenol oxidase) | + | + | + | + | + | − | − | − |
| F. nucleatum | + | + | + | + | + | + | + | + |
| F. nucleatum (polyphenol oxidase) | + | + | + | + | + | + | + | + |
| F. nucleatum (asparaginase) | + | + | + | + | + | − | − | − |

### Example 5

### Asparaginase and Polyphenol Oxidase Inhibit the “Trypsin-Like” Protease Activity of P. gingivalis

As part of the investigation of mechanisms by which these enzymes might act, polyphenol oxidase and asparaginase were evaluated and demonstrated to inhibit a protease activity implicated in adhesion by P. gingivalis.

Materials and Methods

Cell suspensions of P. gingivalis were adjusted up to 1.3 OD at 540 nm in PB. A 100 μl volume of 700 μg/ml BAPNA (benzoyl-DL-arginine-p-nitroanilide) was added to 3 ml of cell suspension. The cells were incubated at 37° C. for different periods of time. The blank contained BAPNA and buffer in the same proportion as P. gingivalis cell suspension. Tubes were centrifuged and the subsequent supernatant was diluted 1:3 and read at 405 nm. Asparaginase and polyphenol oxidase were 20 and 80 U/ml, respectively.

Results

Results clearly showed that the trypsin-like activity is diminished by the polyphenol oxidase and/or asparaginase.

Conclusion

The asparaginase and polyphenol oxidase thus not only affect the adhesion and/or hemagglutination, but also the proteolytic activity of the P. gingivalis. Confirmation of an effect on protease was shown through reduction of the hydrolysis of the collagen substrate (azocoll) asparaginase or polyphenol oxidase.

### Example 6

### Asparaginase Inhibits Adhesion of E. coli to a Mannose Receptor

Materials and methods were generally as described in the Examples above, but modified for observing adhesion by E. coli to a mannose receptor. For example, E. coli possessing type 1 frmbriae were grown and treated with asparaginase according the methods described above for experiments with either asparaginase or polyphenol oxidase. The E. coli were then incubated with a source of mannose receptor.

E. coli that had been treated with asparaginase exhibited reduced binding to the mannose receptor compared to cells that had not been so treated.

### Example 7

### Polyphenol Oxidase Inhibits Adhesion of E. coli to Yeast

To demonstrate broad range of interactions that can be inhibited by enzymes that modify the binding sites of adhesins, polyphenol oxidase was evaluated and shown to inhibit adhesion of bacteria to eukaryotic cells.

Materials and Methods

Materials and methods were generally as described in the previous Examples, but slightly modified for observing adhesion by E. coli to yeast. For example, E. coli possessing type 1 fimbriae were grown in tryptic soy broth (TSB) statically in 37° C. for 18 h. Cells were washed 2×in PBS and suspended in PB. Cells were treated with polyphenol oxidase at 37° C., washed 2×, and resuspended in PBS. E. coli (5×10⁸ cells) were incubated with 1×10⁵ Saccharomyces cerevisiae cells in a 3 ml volume and rotated with an end-over-end motion at 37° C. for 30 min. Suspensions were harvested, washed 2×in PBS and resuspended in 300 μl PBS. Methyl-α-D-mannose was added to some reaction mixtures. Ten ill samples were withdrawn and placed on a microscope slide, heat-fixed, stained with crystal violet for 2 min, rinsed and dried. Preparations were viewed at 1000× with phase-contrast microscopy. Numbers of bacteria bound to yeast cells were tabulated.

Results and Conclusions

Polyphenol oxidase treatment of E. coli significantly decreased its adhesion to mannose-containing Saccharomyces cells. Methyl-α-mannose added to control cells prevented their attachment to Saccharomyces. Preincubating E. coli with methyl-mannose before polyphenol oxidase treatment eliminated polyphenol oxidase mediated reduction of adhesion. Type 1 fimbriae of E. coli are therefore susceptible to polyphenol oxidase, and the mannose ligand is protective against the effects of polyphenol oxidase. Polyphenol oxidase did not reduce the viability of the E. coli.

### Example 8

### P lyphenol Oxidase and Asparaginase Inhibit Adhesion by Helicobacter pylori

Demonstrating the broad range of bacteria for which adhesion can be inhibited by enzymes that modify the binding sites of adhesins, polyphenol oxidase and asparaginase were evaluated and shown to inhibit adhesion of H. pylori to eukaryotic cells.

Experiment 1

Materials and Methods

Methods for handling polyphenol oxidase and observing adhesion by microorganisms were generally as described in the previous Examples with variations to adapt these methods to H. pylori.

Hemagglutination can be determined by any of a variety of methods known to those of skill in the art. In particular, the effect of polyphenol oxidase on adhesion by a microorganism can be studied by a procedure in which polyphenol oxidase-treated bacteria (usually 50 μl) was serially diluted into round-bottom microtiter plate wells. An equal volume of a red cell suspension was added. Controls, no bacteria or untreated bacteria, were run in parallel. Untreated bacteria served as a positive control. The plates were gently rotated (reciprocal motion) for 30 min and aggregates allowed to settle. The titer was taken as the reciprocal of the greatest bacterial dilution giving rise to agglutination. Runs were typically in duplicate. All wells were compared to the negative (no bacteria) control.

A clinical isolate of H. pylori was obtained from the University of Louisville Hospital. The organism had been subcultured on blood agar-Isovitalex in 10% CO₂ and 5% O₂ for 36-40 hr. Cells were scraped from the medium and washed twice with PBS. The bacteria were suspended to an absorbance (1-cm, 540 rn) of 0.8 and a 100 μg/ml final concentration of mushroom polyphenol oxidase (Sigma Chemical Company) was added. The suspension was then incubated 1 hr at 37° C. after which the cells were again washed twice in PBS and suspended to an absorbance of 1.0. Cells treated identically in the absence of polyphenol oxidase served as controls. Human red blood cells (RBC) (group O) were obtained from a volunteer and washed 3× with PBS. The RBCs were used as 3% suspensions.

The bacteria were diluted two-fold into round-bottom microtiter plates, starting at an absorbance of 1.0. To 50 μl dilutions of bacteria were added 50 μl volumes of RBCs. All samples were run in duplicate. Hemagglutination was observed by the appearance of dispersed or roughly settled cells in the microtiter plates. Control RBCs settled smoothly and evenly in the plates.

Results and Conclusions

The results showed that bacteria serially diluted six times could hemagglutinate, whereas polyphenol oxidase-treated bacteria hemagglutinated only to the third dilution. The results, stated in other terms, show that control cells at absorbance of 0.0156 could hemagglutinate, whereas polyphenol oxidase treated bacterial required a cell density of 0.125 absorbance units.

Experiment 2

Materials and Methods

Methods for handling polyphenol oxidase and asparaginase and for observing hemagglutination were generally as described above and in the previous Examples with the following variations.

Two oropharyngeal isolates (one from VA Hospital (Dr. Mary Kemper), the other from UL Hospital (Dr. Jim Snyder), both biotype 1) were grown overnight at 35° C. in 5% CO₂ in shallow BHI medium supplemented with 4% Fildes (Difco) reagent. The cells were centrifuged and washed 2× in the appropriate PB or PBS buffer. After suspension, the cells were incubated with asparaginase or polyphenol oxidase, washed 2×in PBS and assayed for hemagglutination using human group 0 red cells. The suspended H. influenzae had a density of 1.0 absorbance units.

Results and Conclusions

The results of Experiment 2 are shown in Table 5. These results illustrate that treatment with asparaginase or polyphenol oxidase can reduce adhesion by H. influenzae to undetectable levels in hemagglutination experiments for one isolate, and increase titers 4 to 8-fold for another isolate.

TABLE 5
Hemagglutinin Titer of H. influenzae

| VA Hospital | |
|---|---|
| Control | 32 |
| asparaginase (20 U/ml) | 1:4 |
| polyphenol oxidase (100 U/ml) | 1:8 |
| UL Hospital | |
| Control | 1:32 |
| asparaginase (20 U/ml) | 0 |
| asparaginase (75 U/ml) | 1:8 |
| polyphenol oxidase (90 U/ml) | 0 |
| polyphenol oxidase (20 U/ml) | 0 |

### Example 9

### Polyphenol Oxidase and Asparaginase Inhibit Influenza Virus Adsorption to Erythrocytes

Demonstrating the broad range of microbes for which adhesion can be inhibited by enzymes that modify the binding sites of adhesins, polyphenol oxidase and asparaginase were evaluated and shown to inhibit adhesion of influenza virus to erythrocytes.

Materials and Methods

Methods for handling polyphenol oxidase and asparaginase and for observing hemagglutination were generally as described in the previous Examples with the following variations for influenza virus.

Hemagglutination of Chicken Erydirocytes

Influenza A strain H1N1 was obtained from the University of Michigan Department of Public Health. It was propagated in the allantoic fluid of specific-pathogen-free embryonated chicken eggs. Allantoic fluid containing virus was treated with polyphenol oxidase (70 U/ml) or asparaginase (10 U/ml) and tested for its ability to hemagglutinate chicken erythrocytes containing neuraminic acid glycoproteins on their surfaces. Before hemagglutination, the enzymes were removed by ultrafiltration through a membrane with a 300,000 Dalton cut-off. All experiments were performed in triplicate, and controls consisting of incubation with appropriate buffers were conducted.

Results and Conclusions

Inhibition of hemagglutination of chicken erythrocytes by polyphenol oxidase and asparaginase was observed (Table 6). Hemagglutination requires adhesion by the influenza virus. Lower titers indicate a higher concentration or larger amount of virus required to observe adhesion, or weaker/inhibited adhesion.

TABLE 6
Hemagglutination Titers for Enzyme-Treated
and Control Influenza H1N1

| | | | Polyphenol |
|---|---|---|---|
| | | Asparaginase | Oxidase |
| | Untreated | treated | treated |
| Dilution of | 256 | 64 | 64 |
| Virus + RBCs | | | |
| (fold) | | | |

### Example 10

### Polyphenol Oxidase and Asparaginase Inhibit Adhesion by Salmonellae Strains from Chickens

Demonstrating the broad range of bacteria for which adhesion can be inhibited by enzymes that modify the binding sites of adhesins, polyphenol oxidase and asparaginase were demonstrated to inhibit binding by salmonellae isolated from chickens to red blood cells.

Materials and Methods

Methods for handling polyphenol oxidase and asparaginase and for observing adhesion by microorganisms were generally as described in the previous Examples with variations to adapt these methods to salmonellae.

Three Salmonella enteritidis clinical isolate strains, SE 79, SE 89-8312 and SE S1-072-Z were used. The bacterial strains were grown statically in brain heart infusion (BHI) broth for 48 h at 37° C. The cells were harvested by centrifugation and washed twice in 10 mM phosphate buffer (PB) pH 7.5. The final bacterial pellet was then suspended in the same buffer to the desired density.

Bacterial suspensions OD 1.0 (5 ml each) were mixed in the absence and presence of polyphenol oxidase (70 U/ml) or asparaginase (65 U/ml) and incubated statically at 37° C. for 2 h. The cells were then centrifuged and washed 2 times with PB (pH 7.5) and were suspended at a final volume 125 μl. These cells were subjected to hemagglutination tests.

For hemagglutination studies, red blood cells from horse were used. The red blood cells were washed with PBS (pH 7.5) by low speed centrifugation 10 min and suspended to 2% in the same buffer.

Results and Conclusions

Hemagglutination caused by salmonellae strains isolated from chickens was inhibited by polyphenol oxidase and asparaginase (Tables 7 and 8).

TABLE 7
Hemagglutination Effect of Salmonella enteritidis Following
Treatment with polyphenol oxidase or asparaginase

| | Dilutions | | | | | | | |
|---|---|---|---|---|---|---|---|---|
| | 1/1 | 1/2 | 1/4 | 1/8 | 1/16 | 1/32 | 1/64 | 1/128 |
| SE 79 | | | | | | | | |
| Control | + | + | + | + | + | + | + | + |
| polyphenol oxidase | − | − | − | − | − | − | − | − |
| Asparaginase | + | − | − | − | − | − | − | − |
| SE 89-8312 | | | | | | | | |
| Control | + | + | + | + | + | + | + | + |
| polyphenol oxidase | + | − | − | − | − | − | − | − |
| asparaginase | − | − | − | − | − | − | − | − |
| SES1-0072-Z | | | | | | | | |
| polyphenol oxidase | − | − | − | − | − | − | − | − |
| asparaginase | + | − | − | − | − | − | − | − |

+ Hemagglutination
− No hemagglutination
* 2 hr incubation with enzymes

TABLE 8
Titers Following 20 Min Incubation with Enzymes

| SE 79 | |
|---|---|
| Control | 1:256 |
| polyphenol oxidase | 1:32 |
| asparaginase | 1:64 |
| SE 89-8312 | |
| Control | 1:256 |
| polyphenol oxidase | 1:64 |
| asparaginase | 1:64 |
| SES1-0072-Z | |
| Control | 1:256 |
| polyphenol oxidase | 1:32 |
| asparaginase | 1:64 |

### Example 11

### Polyphenol Oxidase and Asparaginase Inhibit Adhesion of Entamoeba to Sheep Blood Cells

Demonstrating the broad range of microbes for which adhesion can be inhibited by enzymes that modify the binding sites of adhesins, polyphenol oxidase and asparaginase were evaluated and shown to inhibit adhesion of amoebic pathogen to blood cells.

Materials and Methods

Methods for handling polyphenol oxidase and asparaginase and for observing adhesion by microorganisms were generally as described in the previous Examples with variations to adapt these methods to Entamoeba.

The Entamoeba species were grown in TYI-S-33 medium at 37° C. for 72 h with 5% CO₂. Entamoeba was harvested by chilling the culture at 4° C. and centrifuging for 10 min at 600×g. Packed amoeba were washed twice with phosphate buffer (16 mM K₂PO₄, 3 mM KH₂PO₄, at pH 7.4). The amoeba were suspended in the same buffer to an optical density of 1.0 at540 nm.

The amoeba were treated with polyphenol oxidase or asparaginase for 90 min at 37° C. For asparaginase treatment the amoeba were suspended in phosphate buffer at pH 8. After treatment, the amoeba were washed twice in phosphate buffer.

The sheep blood cells were washed three times with 0.15 M NaCl. To fix the washed RBC they were diluted with 0.15 M NaCl to 50% (vol/vol) suspension, mixed with 50 volumes of fixing solution (9 mM Na₂HPO₄, 85 mM NaCl, 1% glutaraldehyde) at 4° C. and gently agitated for 30 min. The fixed RBC were washed five times with 0.15 M NaCl and five times with distilled water and finally suspended in 0.15 M NaCl.

Adhesion Experiments

Adhesion was started by mixing 50 μl amoeba suspension with 50 μl RBC (1%) for 30 min at room temperature under gentle agitation. Adhesion was stopped by 2.5% glutaraldehyde fixation. The amoeba were stained with Harris modified hematoxylin reagent for 2-3 min. The adhesion was visualized under a microscope. A minimum of 100 blood cells was counted.

The hemagglutination was carried out in 96 microwell (Nunc) plates which 50 μl of 1% suspension of sheep blood cells fixed with 1% glutaraldehyde and 50 μl of serially diluted cells suspension in phosphate buffer (pH 7.4) were added. The cells were adjusted at 1.04 OD at 540 μm. Entamoeba were treated with polyphenol oxidase and asparaginase in NaCl-free buffer for 90 min and 3 h at 37° C., respectively.

Results

Inhibition of binding by Entamoeba was demonstrated by results shown in Tables 9 and 10.

TABLE 9
Hemagglutination Titers of Entamoeba moshkoviskii.

| | |
|---|---|
| Control | 1:64 |
| polyphenol oxidase, 90 U/ml | 1:256 |
| asparaginase, 20 U/ml | 1:16 |

TABLE 10
Hemagglutination Titers of E. histolytica

| | |
|---|---|
| Control titer | 1:128 |
| polyphenol oxidase, 90 U/ml | 1:32 |
| polyphenol oxidase, 200 U/ml | 1:64 |
| asparaginase, 20 U/ml | 1:32 |
| asparaginase, 60 U/ml | 1:32 |

### Example 12

### Polyphenol Oxidase and Asparaginase Inhibit Adhesion of Type 1- and P-fimbriated E. coli to Urinary Epithelial Cells

Demonstrating the broad range of microbes and substrate cells for which adhesion can be inhibited by enzymes that modify the binding sites of adhesins, polyphenol oxidase and asparaginase were evaluated and shown to inhibit adhesion of bacteria to epithelial cells.

Experiment 1

Materials and Methods

E. coli strains possessing type 1 fimbrial activity and E. coli strains possessing p-fimbrial activity were grown, treated, and assayed according to methods described in previous Examples and known to those of skill in the art.

Urinary epithelial cells (UEC) were obtained by a method known to those of skill in the art. Briefly, the cells were obtained by centrifugation (5000×g) of 200 ml of clean-catch-collected first morning male urine. Cells were washed 3×in PBS and suspended to a density of 1×10⁹ cells/nl in an end-over-end apparatus. After 30 minutes mixtures were collected on polycarbonate filters (pore size=10 μm) and washed with three volumes of cold PBS. Filters were pressed onto glass microscope slides which are then heat-fixed and stained with crystal violet for 2 min. Slides were examined at 1000× with phase-contrast microscopy. For each experimental condition 200 UEC's are examined. Adherent bacteria were quantitated as described in previous Examples.

Results and Conclusions

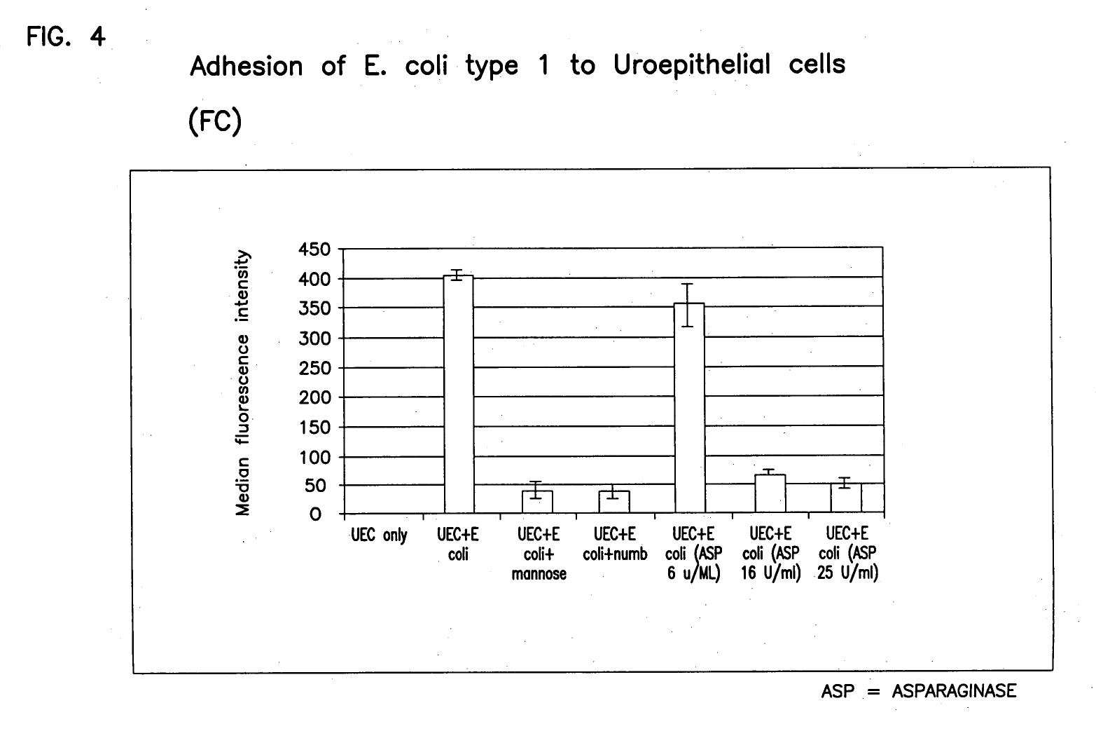

Exposure of the E. coli to asparaginase resulted in reduced binding of these bacteria to urinary epithelial cells (FIG. 4). Greater reduction of binding was observed upon incubation of the bacteria with increasing concentrations of asparaginase or with increasing duration of incubation with asparaginase.

Experiment 2

Flow cytometric analysis of adhesion to demonstrated the inhibitory effect of pretreating the bacteria with polyphenol oxidase and/or asparaginase on their ability to attach to sloughed human urinary epithelial cells (UECs).

Materials and Methods

Methods for handling polyphenol oxidase and asparaginase and for observing adhesion by microorganisms were generally as described in the previous Examples with variations to adapt these methods to epithelial cells.

The bacteria were labeled intracellularly with the fluorescein dye CFDA-SE. Median fluorescence intensity of the UECs was taken as a measure of the number of bacteria attached to them. In the Figures below, median fluorescence is converted to % adhesion using control conditions (UEC+untreated bacteria) as 100%.

Results and Discussion

Type 1-Fimbriated E. coli

Treatment of bacteria with polyphenol oxidase at a concentration of 71 units/ml resulted in little reduction of adhesion (6%), whereas treatment with polyphenol oxidase at concentrations of 141 units/ml and 282 units/ml resulted in greater decreases in adhesion (60% and 44%, respectively) (FIG. 1). The fact that the highest polyphenol oxidase concentration was less effective than the 141 units/ml was seen consistently for polyphenol oxidase treatment of various adhesins. Although not limiting to the present invention, this finding may be due to the formation of Schiff's bases with surrounding proteins which at high concentrations that could include proteins on the host cell membrane.

Treatment of type 1-fimbriated E. coli with asparaginase resulted in more consistent results than those obtained after treatment with polyphenol oxidase. Concentrations of asparaginase <2 units/ml resulted in limited decreases in adhesion (13%); however, concentrations >2 units/ml greatly decreased adhesion (85-90%) to UECs (FIG. 2).

Subjecting the bacteria to sequential enzyme treatments, either polyphenol oxidase followed by asparaginase or vice versa, did not have as great as an effect on reducing bacterial adhesion to UECs as the enzymes did singly. polyphenol oxidase, 141 units/ml, followed by asparaginase, 10 units/ml, resulted in only a 25% decrease in adhesion, while asparaginase, 10 units/ml, followed by polyphenol oxidase, 141 units/ml, gave a 45% decrease in adhesion. Even though these treatments did produce a reduction in adhesion, polyphenol oxidase and asparaginase singly provided much better prevention of adhesion, 60% and 90% respectively (FIG. 3).

To probe the enzymatic site of action, type 1-fimbriated E. coli were incubated with four-methylumbelliferyl α-D-mannopyranoside (MUMB, 50 mM) so as to protect the binding site followed by treatment with either polyphenol oxidase (141 units/ml) or asparaginase (10 units/ml). These treatments resulted in a 25% and 50% reduction in adhesion, respectively. Bacteria were incubated with the mannopyranoside in varied concentrations (10 mM, 50 mM, or 200 mM) then treated with polyphenol oxidase at a concentration of 141 units/ml to observe for a dose dependent effect. The percent of decrease of adhesion remained virtually unchanged (˜30%) for each concentration of the mannopyranoside tested; therefore, 50 mM was used for further assays. The bacteria were incubated with the mannopyranoside (50 mM) followed by polyphenol oxidase (141 units/ml) or asparaginase (10 units/ml), resulting in a 25% decrease in adhesion and 40% increase in adhesion to UECs respectively (FIG. 4).

P-Fimbriated E. coli

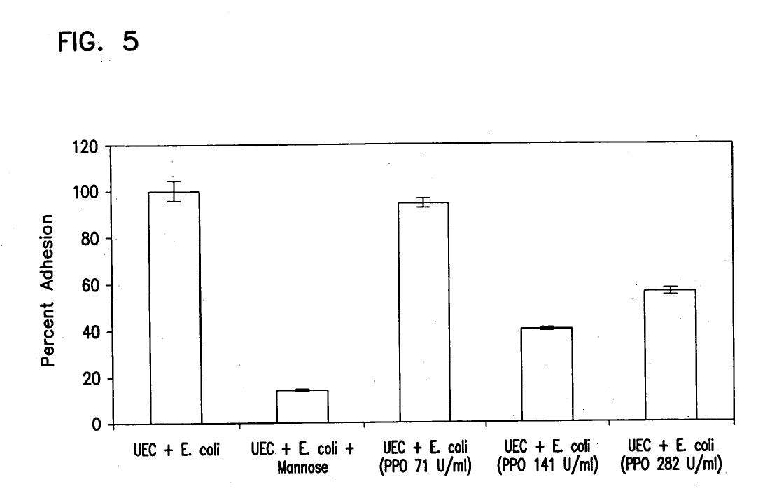

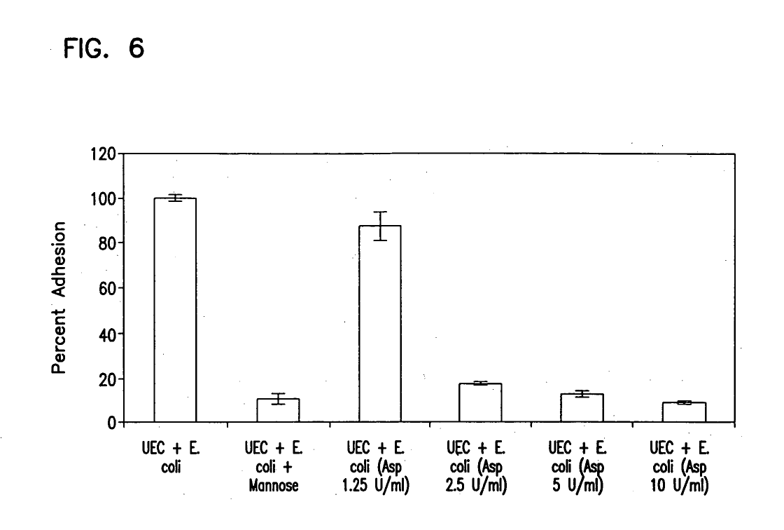

Treatment of bacteria with polyphenol oxidase at a concentration of 71 units/ml consistently resulted in a 40% reduction in adhesion. Treatment with polyphenol oxidase at concentrations of 141 units/ml and 282 units/ml averaged decreases in adhesion of 30% and 55%, respectively (FIG. 5). Treatment of P-fimbriated E. coli with increasing concentrations of asparaginase (2.5, 5, and 25 units/ml) resulted in 45, 55, and 85% decreases in adhesion respectively (FIG. 6).

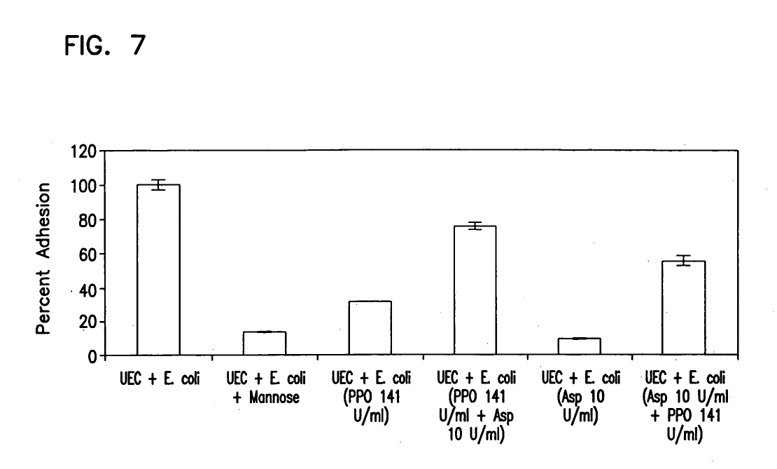

Subjecting P-fimbriated E. coli to sequential enzyme treatments, either polyphenol oxidase followed by asparaginase or vice versa, had varying effects on reducing bacterial adhesion to UECs. polyphenol oxidase, 141 units/ml, followed by asparaginase, 10 units/ml, resulted in a 55% decrease in adhesion, while asparaginase, 10 units/ml, followed by polyphenol oxidase, 141 units/ml, resulted in no decrease from control adhesion (FIG. 7).

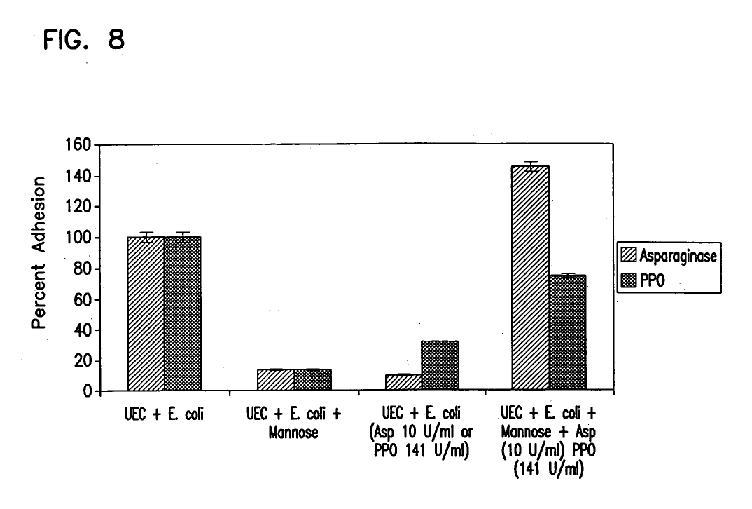

To probe the enzymatic site of action, P-fimbriated E. coli were incubated with globoside to protect the binding site followed by treatment with either polyphenol oxidase (282 units/ml) or asparaginase (5 units/ml). These treatments resulted in adhesion to UECs that was nearly the same as that of untreated bacteria (FIG. 8).

Conclusions

The above results demonstrate that both polyphenol oxidase and asparaginase have the ability to decrease the adhesion of both E. coli types. Decreases in enzyme effectiveness after pre-incubation of bacteria with cognate carbohydrate ligands demonstrate that the enzymes affect the actual binding site on the bacterial adhesins.

### Example 13

### Polyphenol Oxidase and Asparaginase Inhibit Adhesion of Streptococcus pyogenes to Buccal Epithelial Cells

Demonstrating the broad range of microbes and substrate cells for which adhesion can be inhibited by enzymes that modify the binding sites of adhesins, polyphenol oxidase and asparaginase were evaluated and shown to inhibit adhesion of bacteria to epithelial cells.

Methods for handling polyphenol oxidase and asparaginase and for observing adhesion by microorganisms were generally as described in the previous Examples with variations to adapt these methods to Streptococcus and to epithelial cells.

Concentration Dependence of Polyphenol Oxidase Effect on S. pyogenes Adhesion

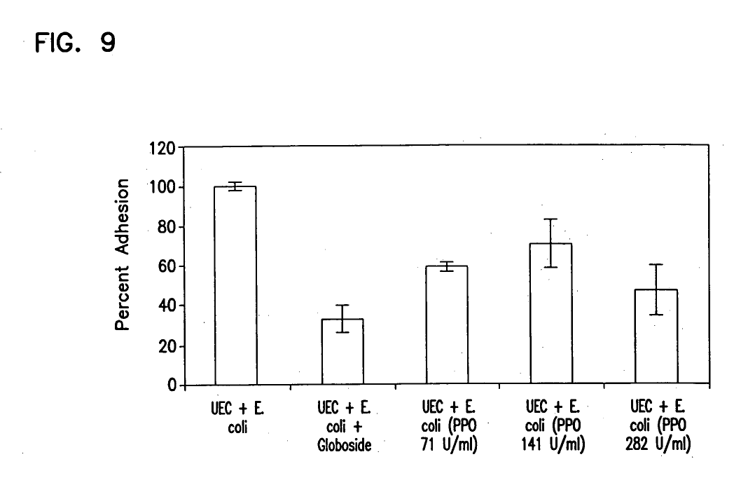

FIG. 9 depicts the relative adhesion of S. pyogenes to buccal epithelial cells as measured using flow cytometry.

Asparaginase Inhibits Adhesion of Streptococcus pyogenes to Buccal Epithelial Cells

This experiment began with growing and harvesting of five strains of S. pyogenes, including M14 Lowe, YL3, M24 and T2/MR. The strains were grown in BHI broth in a 37 degree incubator overnight. The cultures were grown under static conditions. The cells were then harvested by washing twice with phosphate buffer (pH=7). Treatment of the cells with polyphenol oxidase and asparaginase began with finding the optical density of the stock solution of harvested cells. This was done using a spectrophotometer. Each strain was treated with two concentrations of polyphenol oxidase (100 μg/OD and 300 μg/OD) and one concentration of asparaginase (300 μg/OD). The treatment of the cells with polyphenol oxidase took place in the 37 degree incubator for 1.5 hours. The cells were then washed twice with phosphate buffer (pH 7). At this time, a coaggregation was made and allowed to set at static conditions for two hours. Slides were then made of each coaggregation.

The treatment of the cells with asparaginase also took place in the 37 degree incubator for three hours. Before treatment, the cells were washed twice with phosphate buffer of pH=8. After treatment, they were washed twice with phosphate buffer of pH=7. Coaggregations and slides were then made as for polyphenol oxidase.

The buccal cells were harvested from the inner cheeks using cotton swabs. The cells were then washed five times with phosphate buffer (pH=7) before being used in the coaggregations.

The data demonstrated that treatment of S. pyogenes with asparaginase reduced binding of these bacteria to buccal cells. Asparaginase provided greater reduction in adhesion than an equivalent amount of polyphenol oxidase.

Polyphenol Oxidase and Asparaginase Inhibit Adhesion of Streptococcus pyogenes to Human Buccal Cells

Procedure

Streptococcuspyogenes strains were grown and pure cultures isolated on blood agar plates at 37° C. Each were subsequently inoculated into brain heart infusion (BHI) nutrient broth at 37° C. for continued growth.

The cells of each S. pyogenes strains were then washed with phosphate buffer (pH 7) three times. The optical density (OD) of each stain was measured using a spectrophotometer, and adjusted to 0.8. One ml of each strain was treated with polyphenol oxidase or asparaginase. The controls and enzyme-supplemental cells were incubated for ninety minutes at 37° C. Each were washed three times for ten minutes at 15,000 rpm, and recollected in 0.9 ml phosphate buffer.

Human buccal cells were collected and cleaned in phosphate buffer. The buccal cells and S. pyogenes were combined in a ratio of 3:1 respectively, for each strain and allowed to incubate 30 min. Buccal cells were removed by centrifugation (500×g) and washed three times to reduce streptococcal background. The buccal cell-streptococcal complexes were then smeared on slides. The resulting slides were stained using the Gram method, and viewed under the 100× oil immersion lens. The number of S. pyogenes for each strain was counted for at least 50 buccal cells.

Results and Conclusions

The results (Table 11) show that several serotypes of group A streptococci were sensitive to asparaginase and polyphenol oxidase. The results indicate that a mouthwash/gargle including asparaginase or polyphenol oxidase would be useful for sore throat.

TABLE 11
Adhesion of S. pyogenes to Human Buccal Cells^{a}

| | Polyphenol | | | |
|---|---|---|---|---|
| # S. pyogenes | Oxidase | Percent | Asparaginase | Percent |
| buccal cell | (250 U) | Inhibition | (100 U/ml) | Inhibition |
| Strain M+ | | | | |
| 117 | 0 | 0 | 148 | 0 |
| 74 | 100 | 37.1 | 53 | 64 |
| 84 | 300 | 30 | ND | |
| Strain M4 | | | | |
| 220 | 0 | | 183 | 0 |
| 124 | 100 | 43 | 69 | 62 |
| 102 | 300 | 53 | ND | |
| Strain M12 AC | | | | |
| 179 | 0 | 0 | 191 | 0 |
| 113 | 100 | 37 | 89 | 53 |
| 166 | 300 | 7 | ND | |
| Strain M14 | | | | |
| 201 | 0 | 0 | 183 | 0 |
| 126 | 100 | 37 | 105 | 43 |
| 149 | 300 | 26 | 77 | 47 |
| Strain M15 Lowe | | | | |
| 141 | 0 | 0 | 120 | 0 |
| 131 | 100 | 7 | 93 | 23 |
| 132 | 300 | 7 | ND | |
| Strain M24 | | | | |
| 163 | 0 | 0 | 142 | 0 |
| 143 | 100 | 12 | 55 | 61 |
| 124 | 300 | 25 | ND | |
| Strain M24 VAN | | | | |
| 119 | 0 | 0 | 131 | 0 |
| 114 | 100 | 5 | 68 | 48 |
| 116 | 300 | 3 | ND | |

^{a}“M” refers to M-type protein. At least 50 buccal cells were counted for each value shown. Values above were determined by light microscopy.
ND, not determined.

### Example 14

### Polyphenol Oxidase and Asparaginase Inhibit Adhesion of Pneumococci and Group B Streptococci to Buccal Cells

Demonstrating the broad range of microbes and substrate cells for which adhesion can be inhibited by enzymes that modify the binding sites of adhesins, polyphenol oxidase and asparaginase were evaluated and shown to inhibit adhesion of additional bacteria to epithelial cells.

Methods for handling polyphenol oxidase and asparaginase and for observing adhesion by microorganisms were generally as described in the previous Examples with variations to adapt these methods to these bacteria and to buccal cells. In particular, adhesion was measured in a manner similar to that just described for S. pyogenes.

The S. pneumoniae strains were poorly adherent, but asparaginase was able to reduce adhesion of strain 6303. For S. agalactiae (group B) the adhesion was reduced by both enzymes. At least 100 buccal cells were counted.

### Example 15

### Polyphenol Oxidase and Asparaginase Have a Small Effect on Normal Biota Attached To Urinary Epithelial Cells.

Polyphenol oxidase and asparaginase were evaluated and shown to have a disproportionate effect on pathogenic (newly colonizing) bacteria compared to long-term nonpathogenic colonizers (normal biota).

Materials and Methods

Methods for handling polyphenol oxidase and asparaginase and for observing adhesion by microorganisms were generally as described in the previous Examples with variations to adapt these methods to normal biota.

Sloughed epithelial cells were incubated with the enzymes, collected by filtration, transferred to microscope slides and stained with crystal violet. Then 50 epithelial cells from each slide were randomly assessed for the numbers of bacteria associated with them and placed into one of four categories: Cells with 0-9, 10-29, 3049, or >50 bacteria associated with them.

Results

Removal of Endogenous Biota from Slouphed Urinary Epithelial Cells

Treatment with either polyphenol oxidase (70 U/ml or 140 U/ml) or asparaginase (5 U/ml or 10 U/ml) had relatively minor effects on the numbers of endogenous bacteria attached to UECs after a 30 minute incubation with the enzymes.

Table 12 reports the number of UECs (as a percentage of 50 UECs) carrying more than 30 bacteria. The effects of enzyme treatment were concentration dependent. polyphenol oxidase treatment decreased the numbers of UECs with large numbers of endogenous bacteria; asparaginase had little or no effect.

TABLE 12
Removal of Normal Microbiota from Urinary Epithelial Cells by
Polyphenol Oxidase and Asparaginase
Percent UECs with ≧ 30 Bacteria Attached

| | polyphenol | | |
|---|---|---|---|
| | oxidase | | Asparaginase |
| 140 U/ml | 8 | 10 U/ml | 26 |
| 70 U/ml | 18 | 5 U/ml | 28 |
| PBS | 20 | PBS | 30 |

Conclusion

This showing that that the enzymes affect pathogenic (newly colonizing) bacteria disproportionately to long-term nonpathogenic colonizers (normal biota) can facilitate therapeutic use of polyphenol oxidase or asparaginase.

### Example 16

### Polyphenol Oxidase and Asparaginase Inhibit Adhesion by Yeast to Soft Tissue Cells

Demonstrating the broad range of microbes for which adhesion can be inhibited by enzymes that modify the binding sites of adhesins, polyphenol oxidase and asparaginase were evaluated and shown to inhibit adhesion of a eukaryote, yeast, to soft tissue cells.

Materials and Methods

Methods for handling polyphenol oxidase and asparaginase and for observing adhesion by microorganisms were generally as described in the previous Examples with variations to adapt these methods to yeast.

C. albicans was grown overnight at 37° C. in Schaedler's broth. Colonies were maintained on Sabaroud's agar. Hyphae were from same medium but supplemented with 1% Triton X-100. The cells from overnight culture were harvested by centrifugation and washed 2× in PB and finally suspended in the same buffer to a density of 0.8. These cells were then incubated with asparaginase or polyphenol oxidase, washed 2× in PBS, suspended and mixed with washed human buccal cells.

Following incubation, the buccal cell-Candida mixtures were centrifuged at 500×g, washed 3× in PBS and smeared onto slides for staining and counting. Background Candida were disregarded. Only cells directly adherent onto buccal cells were counted. In some experiments, the distribution of candidae was determined and plotted (not shown). In other experiments, enrichment for C. albicans hyphae was realized by incubating the cultures.

Results

Results are shown in Table 13. A minimum of 100 buccal cells for each condition was analyzed for adhesion.

TABLE 13
Adhesion of Candida albicans to Buccal Cells

| Treatment | Candida/buccal cell |
|---|---|
| Control cells | 9.0 ± 3.5 |
| polyphenol oxidase (70 U/ml) | 6.0 ± 1.5 |
| polyphenol oxidase (140 U/ml) | 4.5 ± 1.5 |
| polyphenol oxidase (210 U/ml) | 8.5 ± 3.5 |
| asparaginase (20 U/ml) | 7.0 ± 2 |
| asparaginase (60 U/ml) | 4.5 ± 1.5 |
| Control hyphae | 5.1 ± 3.1 |
| polyphenol oxidase (70 U/ml) | 4.1 ± 2.2 |
| polyphenol oxidase (210 U/ml) | 5.3 ± 2.7 |
| asparaginase (20 U/ml) | 3.1 ± 2.0 |
| asparaginase (60 U/ml) | 1.3 ± 1.2 |

Enzyme incubations were at 37° C. for 60 min. Results in table are ave±SE for 5 separate runs.

### Example 17

### Polyphenol Oxidase and Asparaginase Inhibit Adhesion by Bacteria to Extracellular Matrix Proteins

Demonstrating the broad range of substrates for which adhesion can be inhibited by enzymes that modify the binding sites of adhesins, polyphenol oxidase and asparaginase were evaluated and shown to inhibit adhesion of bacteria to extracellular matrix proteins.

Materials and Methods

Methods for handling polyphenol oxidase and asparaginase and for observing adhesion by microorganisms were generally as described in the previous Examples with variations to adapt these methods to extracellular matrix proteins.

Collagen Binding Assay

Wells were filled with 1 mg/ml soluble collagen overnight at 4° C. Control wells were coated with 1% BSA. The protein solutions were removed and permitted to stand at 37° C. for 1 h. A solution of 2% BSA was added to the wells to block unoccupied sites and prevent non-specific binding of bacteria. After 30 min, BSA was removed and the wells were washed once with distilled water. Finally, the bacterial suspensions (100 μl, OD 1.4) were added to the plates and incubated at 37° C. for 2 h. Unbound bacteria were removed by washing the wells three times with PBS containing 0.01% Tween 20. The wells were dried at 37° C. for 30 min and stained with crystal violet for 15 min. Wells were rinsed with distilled water and dried at 37° C. for 2 h. After adding 100 μl of 95% (v/v) ethanol to each well the plates were then shaken to release the stain and a reading of 550 nm was carried out.

Fibronectin Binding Assay

Wells were filled with human serum fibronectin (50 μg/ml) diluted in carbonate buffer (50 mM Na₂CO₃—NaHCO₃, pH 9.5 overnight). Controls were coated with 1% BSA. The wells were washed three times with PBS and incubated with PBS-Tween (0.5% Tween 20 and 0.05% NaN₃) 1 h at 37° C. After attachment, the wells were washed three times with PBS-Tween and allowed to dry. Subsequent assays were as described hereinabove.

Results

Inhibition by polyphenol oxidase or asparaginase of binding by bacteria to connective tissues was observed and the results are reported in Tables 14 and 15.

TABLE 14
Adhesion of Cocci to Collagen

| Absorbance (collagen) | | Polyphenol | |
|---|---|---|---|
| Bacteria | Untreated | Oxidase | Asparaginase |
| S. pyogenes M4 | 0.19 | 0.14 | 0.10 |
| S. pyogenes M24 | 0.31 | 0.26 | 0.13 |
| S. aureus Wood | 0.40 | 0.25 | 0.26 |
| S. aureus Cowan | 0.47 | 0.36 | 0.30 |
| S. epidermidis (clinical isolate) | 0.09 | 0.06 | 0.05 |
| Control (BSA) | 0.06 | 0.08 | 0.08 |
| Control (uncoated plastic) | 0.06 | 0.06 | 0.08 |

TABLE 15
Adhesion to Fibronectin

| Absorbance (fibronectin) | | Polyphenol | |
|---|---|---|---|
| Bacteria | Untreated | Oxidase | Asparaginase |
| S. pyogenes M4 | 0.32 | 0.21 | 0.15 |
| S. pyogenes M24 | 0.33 | 0.25 | 0.21 |
| S. aureus Wood | 0.42 | 0.32 | 0.35 |
| S. aureus Cowan | 0.33 | 0.21 | 0.18 |
| S. epidermidis (clinical isolate) | 0.17 | 0.06 | 0.16 |
| Control (BSA) | 0.05 | 0.06 | 0.05 |
| Control (uncoated plastic) | 0.02 | 0.03 | 0.05 |

* polyphenol oxidase (80 U/ml) and asparaginase (20 U/ml)

### Example 18

### Polyphenol Oxidase and Asparaginase Inhibit Adhesion of Streptococcus sanguis to Hydroxylapatite

Demonstrating the broad range of substrates for which adhesion can be inhibited by enzymes that modify the binding sites of adhesins, polyphenol oxidase and asparaginase were evaluated and shown to inhibit adhesion of bacteria to hydroxylapatite.

Materials and Methods

Methods for handling polyphenol oxidase and asparaginase and for observing adhesion by microorganisms were generally as described in the previous Examples with variations to adapt these methods to hydroxylapatite.

S. sanguis was grown and handled according to procedures known to those of skill in the art. Hydroxylapatite beads were coated with saliva and washed. Adhesion was measured using [³H]thymidine labeled streptococci. Various cell densities were run but using a single 40 mg weight of beads.

Results

A low concentration of polyphenol oxidase, 100 μg/ml or 150 units, gave reduced adhesion. Asparaginase (20 U/ml) also reduced adhesion. The “cooperative” adhesion (loss of upward slope on the Scatchard plot) was absent following both enzyme treatments. Incubation of cells with enzymes was 1 hr at 37° C.

Higher concentrations of polyphenol oxidase, 500 μg/ml, enhanced adhesion, which is believed to be due to high levels of oxidation followed by Schiff's base formation.

### Example 19

### Polyphenol Oxidase and Asparaginase Inhibit Formation and Maintenance of Bioflims

Adhesion of P. aeraginosa

The effect of asparaginase and polyphenol oxidase on adhesion of P. aeruginosa can be studied using the basic methods described in the previous Examples. In an experiment, using culture supernatants treated with 50 units/ml asparaginase, the asparaginase markedly reduced proteolytic activity on azocoll.

For P. aeruginosa studies, a Robbins device (Kharazrni et al., 1999 Robbins device in biofilm research. Meth. Enzymol. 310:207-215.) will be employed. The device allows the bathing of posts or studs with a cell culture or growth medium at non-shearing rates. The bacteria will be introduced through a port, then sterile medium circulated until enough time has elapsed for biofilm development. The experiment will re-circulate the medium, so asparaginase or polyphenol oxidase will not become prohibitively expensive. The experiment will re-circulate the medium for various times, keeping in mind it will become contamiinated itself. However, the Robbins device allows removal of studs for examination and analysis. Such an experiment will determine whether asparaginase or polyphenol oxidase have effects on P. aeruginosa biofiim development by plotting either CFU or alginate vs. time. Controls (no asparaginase or polyphenol oxidase) will also be run for comparison.

Urinary Tract Bioflims

Organisms such as Proteus mirabilis, Proteus vulgaris and P. aeruginosa colonize medical devices such as catheters. Colonization will lead to encrustation. A model described by Tunney et al. (1999, Biofilm and biofilm-related encrustation of urinary tract devices. Meth. Enzymol. 310:558-566.) will be employed. Briefly: The technique has semblance to the Robbins device but offers the opportunity to study actual catheter materials. Sterile catheter segments will be placed on a sterile glass reaction vessel after which bacterial suspension and artificial urine will be pumped through for about 2 hrs. The system will then be flushed with sterile artificial urine and finally with a supplement of asparaginase or polyphenol oxidase. Segments can be removed, rinsed and analyzed for Mg and Ca by atomic absorption. The amount of metal present is proportional to amount of bacteria on the catheters as well as the urease. The experiments will employ the various catheter materials, such as Percuflex, Siltak, polyurethane, etc.

### Example 20

### Polyphenol Oxidase and Asparaginase Inhibit Vaginal Colonization by Group B Streptococcus

Group B streptococcus is the most common cause of life threatening infections in newborns. The infection is acquired by infants during passage through the birth canal and also during the post-partum period. Inhibiting adhesion of the Group B streptococcus to vaginal tissue can reduce or prevent these infections.

Bacteria. Streptococcus agalactiae (Lancefield group B) will be inoculated into Todd-Hewitt broth (THB) and incubated at 37° C. for 12 h. Cultures will then be washed three times and resuspended in 5 ml of THB to a density of 10⁸-10¹⁰ bacteria per ml.

Rats. Thirty six 80- to 90-day old virgin female albino Sprague-Dawley rats will be housed under standard conditions (25° C.; relative humidity, 40%). Food and water will be available ad libitum.

In vivo infection model. Vaginal infection of Sprague-Dawley rats has been shown to mimic human infection closely (Ancona and Ferrieri, 1979, Experimental vaginal colonization and mother-infant transmission of Group B streptococci in rats. Infect. Immun. 26:599-603.). Rats will be staged in estrous cycle by observation of external genitalia and inoculations will occur on two consecutive days during diestrous. An automatic pipette will be inserted atraumatically into the vagina and 0.1 ml of a suspension containing 10⁷-10⁹ bacteria will be injected into all 30 rats. 36 h and 48 h after the second bacterial inoculation 12 rats will be similarly instilled with 0.1 ml sterile PBS. Twelve rats will be instilled with 40 U polyphenol oxidase in 0.1 ml PBS and twelve rats will be instilled with 8 U asparaginase in 0.1 ml PBS.

Vaginal cultures will be performed immediately before both inoculations with bacteria, and immediately before the first and the second instillations with enzyme or buffer Further cultures will be performed at 24 h intervals thereafter. Vaginal cultures will be obtained by rotating a cotton swab moistened with THB in the vaginal orifice. Five μl of tail vein blood will also be obtained for culture from all rats immediately before the first enzyme administration and 24 h after the second enzyme administration.

Cultures obtained before inoculation will be streaked on tryptose-agar base containing 6% sheep blood. All other cultures will be streaked on plates of Columbia blood agar base containing 10 μg of colistin sulfate per ml and 15 μg of nalidixic acid per ml. Swabs will be vortexed in 0.5 ml of sterile PBS which will be applied to the agar plates.

In all rats displaying a decrease in group B streptococcus colonization over the course of the experiment, separate vaginal cultures will be taken and all aerobic bacteria isolated. Each aerobic colony will be spotted onto lawns of group B streptococcus to test for growth inhibition.

Toxic effects of the treatments will be monitored by observation of vaginal tissues and presence of vaginal discharge.

Statistics. The chi-square test will be used to compare treated and untreated groups at each time point.

TABLE 16
Rat Experiment Timeline.

| Day | | 1 | 2 | | 3 | 4 | 5 | 6 |
|---|---|---|---|---|---|---|---|---|
| Hour | 0 | 24 | | 60 | 72 | 96 | 120 | 24 h |
| | | | | | | | | intervals |
| Culture | Ö | Ö | | Ö | Ö | Ö | Ö | Ö |
| Admin: | GBS | GBS | | Enz | Enz | — | — | — |

### Example 21

### Polyphenol Oxidase and Asparaginase Inhibit Middle Ear Infections by Haemophilus influenzae and Streptococcus pneumoniae

Streptococcus pneumoniae and Haemophilus influenzae are the #1 and #2 cause of middle ear infections (otitis media). Disrupting attachment of these bacteria rather than lyse them through the use of antibiotics presents an attractive alternative for treatment.

Animals. The chinchilla is an advantageous animal model for middle ear infection in which the disease can be produced by very small inoculate injected into the middle ear and in which the disease remains localized to the middle ear in most cases (Giebink, 1999, Otitis media: The chinchilla model. Microbial Drug. Resist. 5:57-72). Thirty healthy adults will be divided into six groups for the studies.

Bacteria. Encapsulated strains of Streptococcus pneumoniae will be cultured in tryptic soy broth and agar for 16 h. Haemophilus influenzae will be cultured on chocolate agar for 48 h in 10% CO₂ Bacteria will be harvested and washed three times and resuspended in sterile saline to a density of 1×10⁹-10¹⁰ cells/ml.

In vivo infection model. Intranasal inoculation of Streptococcus or Haemophilus nearly always results in middle ear infection when subjects have been previously infected with adenovirus or influenza A virus. In the proposed studies, direct inoculation of bacteria into the middle ear will be performed in order to avoid use of coinfection with virus. Animals will be checked for evidence of middle ear infection by otoscopy, then divided into the following treatment groups:

TABLE 17
Treatment Groups for Middle Ear Infection.

| | A | B | C | D | E | F |
|---|---|---|---|---|---|---|
| Infec- | H. inf | H. inf | H. inf | S. pneu | S. pneu | S. pneu |
| tion | saline | polyphenol | Aspara- | Saline | polyphenol | Aspara- |
| Treat- | | oxidase | ginase | | oxidase | ginase |
| ment | | | | | | |

Both ears of each animal will be infused with 10⁸-10⁹ bacteria in a volume of 0.1 ml using an automatic pipettor. Twenty four hours later the animals will be reinoculated. An additional 24 hours later treatment will be administered. Polyphenol oxidase (40 U) or asparaginase (10 U) in 0.1 ml saline or 0.1 ml saline alone will be instilled into both ears of each animal.

Infection outcomes. Before each of the inoculations and treatments, and at intervals of 24 hours after the last treatment, disease state will be blindly evaluated by otoscopy and tympanometry of the middle ear. Signs of tympanic membrane inflammation will be rated on a scale of 0-4+ and used to monitor changes in middle ear pressure, tympanic width and tympanic membrane compliance (Suzuki and Bakaletz, 1994, Synergistic effect of adenovirus type 1 and nontypeable Haemophilus influenzae in a chinchilla model of experimental otitis media. Infect. Immun. 62:1710-1718).

Nasopharyngeal (NP) lavage fluids will be collected immediately before first instillation of treatment solution, and at three-day intervals afterwards. NP lavage is performed by inserting 500 μl sterile saline (in small droplets) in one nare, and collection of the fluid from the contralateral nare. These fluids will be serially diluted and plated on appropriate solid medium (Kennedy et al., 2000, Passive transfer of antiserum specific for immunogens derived from a nontypeable Haemophilus influenzae adhesin and lipoprotein D prevents otitis media after heterologous challenge. Infect. Immun. 68:2756-2765).

Statistics. A log-rank test will be used to compare cohorts for relative time to bacterial clearance of the nasopharyngeal passage, as determined by culture-negative status. A repeated-measures ANOVA will be used to compare the pattern of responses over time for the otoscopy and tympanometry observations.

### Example 22

### Polyphenol Oxidase and Asparaginase Inhibit Influenza Virus Infection in a Tissue Culture Model

Polyphenol oxidase and asparaginase have been demonstrated to be effective in reducing influenza A virus attachment to sialic-acid containing red blood cells. These experiments will extend that investigation to investigate the ability of enzyme treatment to reduce cytopathic effects in cultured epithelial cells.

Cell lines and viruses. Influenza strain H1N1 will be propagated as described in the previous Examples. Hep-2 (human epithelial cell line, used as a model for respiratory epithelium) and MDCK (Madin Darby canine kidney cells) will be used as host cells. Both cell lines will be maintained in minimal essential medium supplemented with 10% fetal bovine serum at 37° C. in a humidified atmosphere in 5% atmospheric CO₂ and subcultured twice a week. Virus titer will be determined on both cell lines using a plaque reduction assay.

Cytotoxicity. Cell lines will be observed for cytotoxic effects of the enzyme treatments as follows: Cells will be seeded in 96-well culture plats at a density of 3.5×10⁴ cells/well. After incubation for 16-18 h, various concentrations of polyphenol oxidase and asparaginase will be added to quadruplicate wells and incubation continued for 48 h. A tetmzolium solution (MT) will be added at a concentration of 5 mg/ml and the wells will be incubated for a further 2-3 h. Culture medium will then be removed and DMSO added to dissolve formazan crystals formed by the cellular reduction of MTT. The absorbance (wavelength=570 nm) of each well will be measured. Cytotoxicity is expressed as 50% cytotoxic concentration (CC₅₀) of each enzyme tested.

Inhibition of cytopathic effects (CPE). The 50% cell culture inhibitory dose (CCID₅₀) of virus will be determined. Virus will be diluted with serum-free MEM to the CCID₅₀ and added to confluent cells in 96-well culture plates. Culture medium containing polyphenol oxidase (70-280 U/ml) and asparaginase (10-40 U/ml) will be added immediately to quadruplicate wells. Plates will be incubated for 1-3 days and the MTT assay (above) will be performed. Fifty percent effective concentration (EC₅₀) will be calculated with the following equation: (((ODₜ)ᵥ−(OD_{c})ᵥ)/((OD_{c})_{mock}−(OD_{c})ᵥ)×100), where (ODₜ)ᵥ is the OD of the cell, treated with virus and substances, (OD_{c})ᵥ is the ID of the cell treated with virus control, and (OD_{c})_{mock} is the OD of the mock infected cell. Antiviral activity will be expressed as the value of CC₅₀ divided by 50% effective concentration (EC₅₀, above)

Plaque reduction assay. Confluent host cells grown in 24-well plates will be infected with virus to give 100-200 plaques per well. The plates will be incubated at 37° C. in 5% CO₂ for 1 h with intermittent rocking. Wells will then be overlaid with agar overlay medium containing enzymes at the above concentrations or buffer alone. After 1-3 days incubation, wells will be fixed with 5% buffered formalin, stained with 0.05% crystal violet, and the number of plaques counted. The degree of inhibition will be expressed as yield of control, and the values of EC₅₀ will be calculated by regression analysis (Eo et al., 1999, Antiviral activities of various water and methanol soluble substances isolated from Ganoderma lucidum. J. Ethnopharmacol. 68:129-136).

### Example 23

### Polyphenol Oxidase and Asparaginase Inhibit Infection of Chickens by Salmonella enteritidis

In terms of food-borne illness, Salmonellae contaminated eggs have been implicated more than any other source as causing symptoms. Chicks that acquire S. enteritidis have the bacterium for life, leading to egg contamination. Because we found that the hemagglutinin of S. enteritidis strains was sensitive to asparaginase and polyphenol oxidase, we propose to study the use of the enzymes to reduce or eliminate the bacteria from chickens.

Bacteria. We will use a chick isolate of S. enteritidis that can be obtained from Prof. Peter Holt, USDA, Athens, Ga. The strain is resistant to nalidixic acid and is phage type 13. Tetrathionate brilliant green agar containing 20 μg/ml nalidixic acid is used for plate counts and is selective for the S. enteritidis.

Chicks. New hatched Single Comb White Leghorn chicks will be housed in groups of 10 in a disease-containment area. The chicks will be inoculated orally with exponential S. enteritidis (approx 7.5×10⁶ CFU per 1 ml dose). All birds will be provided feed ad libitum and sterile water (±asparaginase or polyphenol oxidase at 100 μg/ml). Conditions will be as described by Gast and Holt (1998, Persistence of Salmonella enteritidis from one day of age until maturity in experimentally infected layer chickens. Poultry Sci. 77:1759-1762).

Microbiology. Chicks will be sacrificed by cervical dislocation and ceca, livers and spleens examined for CFU on the selective agar. In some experiments, eggs will be examined on CFU when the hens are 21-24 weeks old. In addition, voided feces will be assayed for the bacteria. Controls (no enzyme, will be run in parallel). Based on Gast and Holt (supra) the internal organs should be free of bacteria by 8 weeks, but feces will continue to be culture positive. If enzyme reduces the counts significantly, then a study will be performed solely on salmonellae-chick-enzyme relationships, utilizing enzyme in water and in feed.

In the initial studies, enzyme treatment will be started upon arrival of the chicks. The animals will be housed in groups of 10. Bacteria will be introduced one day later, followed by assay of feces for S. enteritidis. The chicks will be sacrificed at day 5 post-inoculation and ceca, liver and spleens assayed for the bacteria. If bacteria are not found in the internal organs, it will suggest that a supplement of enzyme early in life would suffice to render a flock salmonellae free.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

We claim:

1. A method of reducing binding of a microorganism to a surface, comprising enzymatically modifying an adhesin on the microorganism.

2. The method of claim 1, wherein enzymatically modifying comprises contacting the microorganism with a polyphenol oxidase, an asparaginase, or a combination thereof.

3. The method of claim 1, wherein the microorganism comprises a prokaryote, a eukaryote, a virus, or a combination thereof.

4. The method of claim 3, wherein the prokaryote comprises a gram-positive bacterium, a gram-negative bacterium, or a combination thereof.

5. The method of claim 3, wherein the prokaryote comprises a Staphylococcus.

6. The method of claim 3, wherein the eukaryote comprises a fungus or protozoan.

7. The method of claim 6, wherein the fungus comprises a Candida.

8. The method of claim 1, wherein the adhesin comprises a lectin.

9. A method of reducing adhesion by a microorganism, comprising exposing the microorganism to an effective amount of an enzyme which reduces adhesion by a microorganism.

10. The method of claim 9, wherein the enzyme catalyzes a reaction for modifying a molecule on the microorganism.

11. The method of claim 9, wherein the enzyme catalyzes modification of a side chain of an amino acid.

12. The method of claim 11, wherein the amino acid is found in the binding site an adhesin.

13. The method of claim 11, wherein the amino acid comprises asparagine, tyrosine, or a combination thereof.

14. The method of claim 9, wherein the enzyme modifies a carbohydrate binding site on the microorganism.

15. The method of claim 12, wherein a lectin comprises the carbohydrate binding site.

16. The method of claim 9, wherein the enzyme comprises a polyphenol oxidase, an asparaginase, or a combination thereof.

17. The method of claim 9, wherein the microorganism comprises a prokaryote, a eukaryote, a virus, or a combination thereof.

18. The method of claim 17, wherein the prokaryote comprises a gram-positive bacterium, a gram-negative bacterium, or a combination thereof.

19. The method of claim 18, wherein the prokaryote comprises a Staphylococcus.

20. The method of claim 17, wherein the eukaryote comprises a fungus or protozoan.

21. The method of claim 20, wherein the fungus comprises a Candida.

22. A method of treating an animal, comprising administering to the animal an effective amount of an enzyme which reduces adhesion by a microorganism to the animal's cells or tissues.

23. The method of claim 22, wherein the enzyme comprises a polyphenol oxidase, an asparaginase, or a combination thereof.

24. The method of claim 22, wherein the microorganism comprises a prokaryote, a eukaryote, a virus, or a combination thereof.

25. The method of claim 24, wherein the prokaryote comprises a gram-positive bacterium, a gram-negative bacterium, or a combination thereof.

26. The method of claim 24, wherein the prokaryote comprises a Staphylococcus.

27. The method of claim 24, wherein the eukaryote comprises a fungus or a protozoan.

28. The method of claim 27, wherein the fungus comprises a Candida.

29. The method of claim 22, wherein administering the enzyme comprises oral or topical administration.

30. The method of claim 29, wherein administering the enzyme comprises topical administration to a nasal tissue.

31. The method of claim 29, wherein administering the enzyme comprises oral administration to a digestive tissue.

32. The method of claim 31, wherein the oral administration to the digestive tissue comprises administering a sustained release formulation or an enteric formulation.

33. An oral care composition comprising an effective amount of an enzyme which reduces adhesion by a microorganism.

34. The oral care composition of claim 33, wherein the enzyme comprises a polyphenol oxidase, an asparaginase, or a combination thereof.

35. The oral care composition of claim 33, wherein the microorganism comprises a prokaryote, a eukaryote, a virus, or a combination thereof.

36. The oral care composition of claim 35, wherein the prokaryote comprises a gram-positive bacterium, a gram-negative bacterium, a protozoan, or a combination thereof.

37. The oral care composition of claim 35, wherein the prokaryote comprises a Staphylococcus.

38. The oral care composition of claim 35, wherein the eukaryote comprises a fungus or protozoan.

39. The oral care composition of claim 33, further comprising a buffer, a peroxide, a source of copper ion, an oxygen generating compound, or a combination thereof.

40. The oral care composition of claim 33, wherein the oral care composition comprises a mouthwash, a toothpaste, an implant, or a combination thereof.

41. The oral care composition of claim 33, wherein the oral care composition comprises a solid, a semi-solid, or a liquid composition.

42. A method for reducing adhesion by a microorganism to oral tissues or cells, comprising exposing the oral tissues or cells to an oral care composition comprising an effective amount of an enzyme which reduces adhesion by a microorganism.

43. The method of claim 42, wherein the oral care composition comprises a mouthwash, a toothpaste, an implant, or a combination thereof.

44. A method for reducing adhesion by a microorganism to a dental prosthesis, comprising exposing the dental prosthesis to an oral care composition comprising an effective amount of an enzyme which reduces adhesion by a microorganism.

45. The method of claim 44, wherein the dental prosthesis comprises a denture.

46. A method of making an oral composition useful for reducing adhesion by a microorganism, comprising the step of adding to an oral composition an effective amount of an enzyme which reduces adhesion by a microorganism.

47. A pharmaceutical composition comprising an effective amount of polyphenol oxidase which reduces adhesion by a microorganism and a pharmaceutically acceptable carrier.

48. The pharmaceutical composition of claim 47, wherein the polyphenol oxidase comprises polyphenol oxidase isolated from a microorganism or plant.

49. The pharmaceutical composition of claim 47, wherein the microorganism or plant comprises a thermophilic microorganism, a thermophilic fungus, or a mushroom.

50. The pharmaceutical composition of claim 47, wherein the polyphenol oxidase comprises recombinant polyphenol oxidase.
